SEQUENCE LISTING (14 sequences)

SEQ ID NO: 1 - protein, 293 aa, Homo sapiens
  ref <308>: DATABASE ACCESSION NUMBER: GenBank / AAF03101
  ref <309>: DATABASE ENTRY DATE: 2002-04-16
  ref <313>: RELEVANT RESIDUES: (1)..(293)
MATARPPWMWVLCALITALLLGVTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 2 - DNA, 1387 bp, Homo sapiens
  ref <308>: DATABASE ACCESSION NUMBER: GenBank / AF168768
  ref <309>: DATABASE ENTRY DATE: 2002-04-16
  ref <313>: RELEVANT RESIDUES: (1)..(1387)
ggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacttggggaaatcaggtgcagcggccatggctacagcaagacccccctggatgtgggtgctctgtgctctgatcacagccttgcttctgggggtcacagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaaagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaagtggaaaaaaaaaa

SEQ ID NO: 3 - DNA, 11570 bp, Homo sapiens
  ref <308>: DATABASE ACCESSION NUMBER: GenBank / AF135028
  ref <309>: DATABASE ENTRY DATE: 2000-06-27
  ref <313>: RELEVANT RESIDUES: (1)..(11570)
gggcccagagtgaaggcaagagaaggagttgagagctccctctgcaaagtggcttgagtctcccctgcctaaaatgcagggagagggaggcagaaagacagggaagaggaaggggtggggaagaaagagagagagagagagagacagaataacacaactacagaaacacagagagaacacacagagagcctgggacacagggacacacagagtcagagagaaaagagaagatagagaaagacacaaatggagacacagaggtgtaaagaaagagagattaacagagtcccagatacacgcaaaggggcagaagcacagttttcagggtggtgtctatgatcatcttcttttttttttttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaatttttttgtatttttagtagagacggggtttcaccgttttagccgggatggcctcgatctcctgacctcgtgatccgcccgcctcggcctcccaaagtgctgggattacaggcgtgagccaccgcgcccggccatgatcatcttcttgactatgctgatgtgacaagtacctaaagccatcagactctaccctttaaatatgcagtttgggccaggcaccgtggctcatgcctgtaattccagcactttgggaggcagaggtgggtgaatcacttgaggccaggagtttgagaccagcctggccaacatggtgaaactctgtctttactaaaaaaaaaaaaaaaaaaaaaaaaatcagccgggtgtcgtggggcacacctgtaatcccagctatgctggaggctgaggcacgagagtcacttgaaccctggaggcggaggttgcagtgggccgagatcacatcaccgccctccagcctgggcgacagagcaagactctgtctcaaataaataaataaacaaacgaacaagcagtttgttgtaccttagttatatctaaaaaaaaaatgctgtcaacaaatagagcagaagtgaaataaaggaaaataaatgggccaagaactctaaggtatatttgacaaatcattcagaacctttaaaaaagaaagaatcacagaggcatagaaagacagggaggaacagggagacagaaacacctgtggcccaaggagaacaaaacaaggctcctaagacagacaggaggagagagagagagagtgagtgagagacagacagagaaaaagacagagagagagagacagagacagagagacagagaggcgagagggatagaaagagagagaggggtggagagagacacgagatattgagagagactcagaaagatagccgagggagaaccacagagagatggaagaagactctgagaaaaaaccagagacaaagatggaaagaggagtatcgagggtgaacagacagtggtggaatgagcaaaatgcagagaagaaagcaagcaatccaggcgccaagaatagtgacccagagttggtgagaagccagatccttaaggctgggggaggcagggaaggggctggcctggcttccggagacccctccccattctccgggccagggaggtagggagtgacattccggactgggtggggggtgctctgggggtggagatagggggagcaggaggagctattgctaaggcccgataggcacctcattgcccgggaatgtgccccagggagcagtgggtggttataactcaggcccggtgcccagagcccaggaggaggcagtggccaggaaggcacaggcctgagaagtctgcggctgagctgggagcaaatcccccaccccctacctgggggacagggcaagtgagacctggtgagggtggctcagcaggcagggaaggagaggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacctggggaaataaggtaggggagggaggggaagtgggttaagggctccccggatcgcctgggcctcccaaccctctgacattccccatccaggtgcagcggccatggctacagcaagacccccctggatgtgggtgctctgtgctctgatcacagccttgcttctgggggtcacaggtaaccagaactctggggtgggagggttgtgggattgggaggactgtctctgcggcactagagcgcctgtcccctggggaactgtgtgagcctgggcatgactccgggaccgggtgaatgtgagtctctgtctgtacttgtggttgtgcgatcgtatgtggccctgtgactgccacggtgtgtgtcggggagggggatgccttttcccatatcaggtgactgtgcggcaggtggcactgaccctttgaggctgtgtgtgtggttttgtgattgtgtgtgcatttaagattgtgtgtggctccacagctgtgtgggtgaatgcatgtagcactgggggtgttcactgtgtgtttggctgtgtgtggtgacttggcattgtatatgactgcaggtatctgcagttcctgtccctgaggtcccgggattgcgtgcaacaaaagtggtcatcaccatggaaagctgtgactgtgtgctgcttgcaggcgattatgtgattgtggctgagtgtgacgttatggatgcccgtatttgtgaccgtgtgactacctgaagctctgtgtaggggtgactgtatgtgactgtgtgtgtctgtgtgaggccgtgtaaatgctactgtatgtgtgatggtgcagctgtgtgtctggagtttctgtctctgcctggagggatagagggtgcaggggtagctatctctgggagatgggtgccaggtgactgacttgcagtgtgtgcctgtgtgcagaagagtatgtggcagtctgaacatctgtgcacacacggcatctgtgcgtggcactgagacactgtggatgagggtgtgcgatcccgctaggctgcccgggagcgtgtgtacctggagacagagctgtatgttagctgcacctgtggaggcaacatgggcgtgtctgcagaactgcgtgcgtgcttggctgttactgctgttgtgcgcgtggttcttggggtgagttcgtgaatgatggtggtgccagggccatcagcaagggtaagaaccaggccgggcgcggtggctcacgcctgtaatcccagccctttgggaggccgaggcaggcggatcacctgaggtcgggagatcgaggccagcctgaccaacatggagaaccccgtctctactaaaaatacaaaaaattagctggtgtggtggcgcgtgcctgtaatcccagctactcgggagactggggcagaaaaatcgcttgaacccgggaggtggaggttgcggtgagccgagatcgcgccattgcactccagcctgggcaacaagagcgaaactccgtctcgaaagaaaaaaagaaaaaaaaaagggtaagaaccagtgaatgggcacgggaggactgatgatggagtggggcatgcatgtagtctgtaggtctgtgtgtgagaggaggagattgacaggattgagaaggcatgttttcatctgagaattcagaaacctaggcctgctcttcccctccatgtggccccctaagctgagcccttctttcctggtcctgctttcggaaccctagctccgcccatgagctctgaccccacctcctttcctcaaccacgcccctaggccagactctagtggaccccgcctaaggccacacccctttgggccaggctccaccccctattctgtgggtaccttctagaacccccttcaaagtcagagcttttttttttttttttttggagacagtcttgctctctctcccaggctggagtgcagtggcgtgatctcggctcactgcaacctctgcctcccaggttcaagtgattctcgtgcctccacctcctgagtagctgggattacaggtgcgcgccaccacgcctggctaatttttgtgtctttagtagagacagggtttcaccttgttggccaggctggtctcaaactcccaacctcaggtgatccgcccacctcggcctcccagagtgctggggttacaggcgtgagccaccgcccccagcccaaagtcagagctctttataggagactctaacatgtaaccctgaccctggccctaactaagtcaattccaaaccccttcctgcctccagccctgaccccactcactgaggcctgaccccacttcttgagaccagttccatccctaaagccctggtctccctcccatccccaggctccagcccccacagctttggcactacccctgagcttgtccaggaatcctgtacccaattttaccctcacatgtagttctagccaattccaggaatctgtgaggtccagttagagtccagtaaccctacctgagcctgggctctgtccttgagcttgagcctgggcttgagaggtgccactcttattctccaggccctgcccctgccccctcagcatgtcagacacccaccctctagctggtctggcctcttgagtctgaaacccacccccagcccaagccccgcctctgagccccgcccaacccattttccgttcccagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaagtgagtgggagttccaagaggagggttggtggggacggggaagtgggggtgggggtggggaagtgggggtgggggtgtcatggaggtgagggctggtggggacggggaagtggggttgggggtgtcatggaaggtgagggttggtggggatgggttggggatgtgggagcaggaggaggtcgagttggggataggactaaggatggagttttgcgggggagcaaggtgggaggatgaggttggagaggggagagtgttgtggtagggaatgggaaggagccaaggatgggttggatttggggttaggagcatatatttgttgaatggtttgggatggaggtggaattgggattggctttagaattgggggtgggtgaaaatcgggctggggtggaaatgaagatagcatggagatagggttgagattgggagcagatatagaatgaaggatggggattggagttttgggtggggttggagatggttggatttgggcttgagaatgcatatggtgatggcttctgggtagggaaagaattagggttgggaatgggatgggtttggaattgtgactgggatggggacaggcatgggattggagaccaagagggagttgaggatggtttggggaccgggggtggggatgggggtggggctggggctgggtgtggggttgggattggcgttggacgtggagatagagatcagggttggtggtgacctgccccatcttcctcagagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaaggtgagtgtccaggttcttcttgataccgacccatctctgccgccttccatctttctccacttctcattgtgttcctgtttgacagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccaggtgaggacacctctctttattcagcagatacacactgagtgccaactcggtaacatggagcgttgccaaattctgagaatccagcaattgccaagacagtcaggacccctgttctcacagagctcataccctagagtagtggtgtttagtagaaataatgctgagctgcttatgtcatttccagttttttagtagccacattaaaacaggtaaaaaaggctgggcgcagtggctcacacctgtaatcccagcactttgggaggctgaggcaggcagatcacctttggtcaggagtttgagactagcctggccaacatggcgaaactctgtctctaaaaaaaaatacaaaaattagcctggcatggtggcgggcgcctgtaatctcagctgctcaggaggccgagacacaagaatcacttaaacccaggaggtggaggttgcagtgagctgagatcgtgccactcactccaacctgggagacagagtgacacttttgtctcaaaaagaaaaaaaaaaacaagtaaaaaagaaacaggtgaagttaactttaataacccaatgtatcccaaatacaatcatttcaaagtgtaattaatataaaacaattatgaatgagatactttacattcttttcttgttttcatattaagtctttgaaagtgagtatatatgttatgctgacagcacatctcaatttggactagctacatttcaggtgctcagtagccacatgtggctagcagttactgtattggatggcacggatctagagggaaagatcagggctgttttgtatggttgggcaggttgtgcactgcataaagataccatatctaataggggcactccgtgttacagatgtcagttttggcagttttcaggcgtgtggtagttaagtgtcttgtttcaacaaaatctgtaatatgacagttttctagcaagtgctggtaaaatatcttgaggaaggaaaagagaaatctggtaggtatttttacaagagaatatttaatacaggggattaattgcaaagctgctggaagggctggaggaacaaagttaaaaaataaaaaactctgtggtcaagaatctgcataaatagggcaatttcagagagtggtaaaggttaaccccaaaataaaacatggttttaggatagtaaacaataagggccaatattcaaaaaggtggtcaggggagcctccttggagaggtggcatttgagcagagaatggatgacacaaagaagctaaactcgtgaagtttaaggggaaagaaaaggcacgtgcaaaggccctgaggcagtaaggaatttggctgattcaaagaagaagaggaaaccaatgcaactggagaacaaaagtgggggcaacagtagaaagtgacgctggaggtgtaggcaggggcgaatgctctgcaagtatttcttggtcaccaacacagagcttccctatgttctaatggaagctgtatctgttgaggaagacagaatttaaaatcaaactgttacatcaaccagcacccttctctgtattcaggctcccaagggatctagaaggacgtaagttaacaagctctcattagcagggtgtgtgtttcaacagtagttaggaagctggggattcaggagtactccagtcccatggctatgaaaagctccccccaaattgtacaaacctgacaaatgcaacacctccccagctctccccatttcttctctgtgccctgggtgtgggggggtgggttgcgagggggaaaacttttaacagaagaaagcacatctcggccgggcgtggtggctcacacctgtaatcccaacactttgggaggccgaggcgggtggatcactaggtcaggagatggagaccatcctggctgacacggtgaaaccctgtctctactaaaaacacaaaaaattagccgggcgtggtggcaggcgcctgtagtcccagctactcgggaggctgaggcaggagaatggcctgaacccgggaggcggaacttgcagtgagccgaggttgcaccactgcactccagcctgggcaacacagtgagactccgtctcaaaaaaaaaaaaagaaaagaaaagaaatcacatctcattcaagtggtggcatttaaaactatttagcctttctgtaggcaaggttagtatcttgtttttccagacctcaaggtgtttttttgtttgttttttcataccggtgtgtggtctgggtgtggccactaaaagctacaagcaagaaataataacaactacaacaatactaataccaatagtataaaaataatagcatctggctaattgctggacactgttttaagtggtttgcatgcctcagctcattaactcatttacctgttattattggccctattttacaaacaaggagccaaggctcagagcagttaactaacagcctctcaaaagaaactctgcagagatattaaatttaaaaaataatgagagaaattaaaccacaagaaagttgaaatttagaggtacaggcagctaagcttgtttgctttgaaacagtgtctgctactgggaaaaaggcaagtcttggctttcctaataattgataccaggactctgtaattcatattttgcatgcatgtaagtaagaaatgaagccgggtgcaatggcacatgccagtaatcccagcactctgggagactgaagtgggaagatcacttgagctcaggagttcaagaccagcctgggcaactaaaaattaaaaaaataaaaatactaattgtttttattttagtagattttattcataccacttacatcattattgtagtatgtacatatttatttcttttcttttcttttcttttcttttttgagacggagtctcgctctgtcacccaggctggagtgcaatggcaccatatcagctcactgcagcatgcgcctcctgggttcaagcatttcttccacctcagcctcccaagtagctgggataacaggcacccaccaccatgcctggctatttttttttttccgtagagatggggttccaccatgttggccaggctggtcttgaactcctgacctccagtgatctgcctgcctcggcctcccaaattgctggtattacaggtgtgagccaccgtgcccaggtgggagatagacatttctctctacctcaaacagaggtccactcaagctacttttcattttcttcataaatattagccgagtggctattttgcaccaggaatggttccaggtgctgtggatatggcatcaggcaaaacagaccaaaaacttcctgccgcgtggacctcatgttccccaagtggaagacaggcaataaagagatagataaatatgtagtaaattaaaaaaaaaaaaaattagccgggtgtggtggcttgcacctgtagttccagctacttgggaggctgaggtgggagaattgcttgagcccaaacgtttgaggctgcggtaagccatgactgcactgctgcactccagacagcagcctgggtgacaaagcaagacgtttttgtcagaaagaaaaaaaaaagagacgaagggaggaaggagagagaaaggaaggaaggaaggagaaagaaaggaaggaaggagaaagaaaggaaggaaggaaggagaaagaaaggaagaaagagaaagaaagaaaaagaaagaaagaaagaagaaagaaaagagagaggaaggaaggaaagaaggaaaagagggaaaaaaatgactgttgaagagcagtgagtattattataggagggtaattatagggaggtatggggaattgaagacaggaaacacaaattagtccaagcgaatggatttctattgggagtgattctgcccctagaagacactggcaataccaggagacatttttggttgtcacaactatatggaggggcattactggcaactaatggatagatgccaagtgtgctgttcaacatgctatgatgcacacggcaggcctccacaacaaaccattatccagcttcagatgcccacagtgcccagatcgaggaaccctcatccaggggctgagaaccgtatttttgcagaagggaggtataaggatgggttggtggagaatggggaaggaaggtgtgtgtccagtaagagaaataaggcctgcacaggctggaggggagagtgagagagaaagggaggcggagagatacacgatgagggagacaggctggaacagaaagtagagacgaagattcgagatgtggagaggaagggtcacagacccccccgaaatgatgtgtggacaacaggaatctggaagaggaagatggagtggagagtgacaaatggggtctaaaggttgaacttggaggccaggcatggtggctcacgcctgtaatcccaacactttggaggctgaggtgggcgaatcacttgaggccaggagttcgagaccagcctggccaacatggtgaaaccccgtctctacaaaaaaaatacaaaaaattagccgggtgtggtgatggacacctgtagtcacagctacttgggaggctgaggcaggagaattgcttgaacccgggagatggaggctgcagtgagctgaggtcaggccactgcgctccaacctgggcaacagagtaagactccatctcaaaaaaaaaaaagctggatttggagtgaaatattaataacattctccctctctctccttttgcctgtgtctccatctctgtctttttctgcatttcttcatctctgtactttccatctctgtgtgtctgttcccatctgcttctccatctatgggcatctctgggtctctcatgtctccttctgcccactttgccacatctctgcctctctcatgcccccctttctctcctgcagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaag

SEQ ID NO: 4 - protein, 244 aa, Homo sapiens
  ref <308>: DATABASE ACCESSION NUMBER: GenBank / AAB66483
  ref <309>: DATABASE ENTRY DATE: 1997-08-12
  ref <313>: RELEVANT RESIDUES: (1)..(244)
MKKLMVVLSLIAAAWAEEQNKLVHGGPCDKTSHPYQAALYTSGHLLCGGVLIHPLWVLTAAHCKKPNLQVFLGKHNLRQRESSQEQSSVVRAVIHPDYDAASHDQDIMLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAK

SEQ ID NO: 5 - DNA, 1451 bp, Homo sapiens
  ref <308>: DATABASE ACCESSION NUMBER: GenBank / AF013988
  ref <309>: DATABASE ENTRY DATE: 1997-08-12
  ref <313>: RELEVANT RESIDUES: (1)..(1451)
gtcgacccacgcgtccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaattttattatgaaaaaaaaaaaaaaaaaaaaaaaaaaaagggcggccgc

SEQ ID NO: 6 - DNA, 12483 bp, Homo sapiens
  mRNA (2001)..(2185)
  mRNA (3084)..(3135)
  mRNA (3559)..(3606)
  mRNA (4346)..(4502)
  mRNA (8122)..(8369)
  mRNA (9791)..(9927)
  mRNA (11805)..(12483)
  ref <308>: DATABASE ACCESSION NUMBER: GenBank / AF149289
  ref <309>: DATABASE ENTRY DATE: 2000-06-26
  ref <313>: RELEVANT RESIDUES: (1)..(12483)
acacttaaaaaatcttctgacttaaaaaaaaaagtatggtgattggaaaatgtaaatgtgcatgcgtgcttggcatcacatttcattggccaggacttccctggatgctaaaggtcctcaaatgccaggctggggggctgggacttggtcccaagggagatggggacccagggcacgtctgtgagaggaggggcaaggtcagcacaaggcacaggaaggtctctctggggcaagggatacagagaacagagggatcctggtccaggtgggagaggtgcagctctgagttggggttgagggtgtgggtacagagaggaagggaccccccagagagaggaggcagagggatagggcctggtcactgggttgtgcaacatcagacttgctgtctgtgaagatagcacgtcctgagaagaaggtgctgaggtcagtggggaccaaatgtgagagggagcacccggagagtatactgaataccgaagtagtcttcatccctggagtgatggggggtgcacaatgcaagatgacaattagattcaatgcaagacaaagaaaagggttggctgggaacagtggctcatgcctatggtcccagctcctgggaagactgaggcgggagggtcgcttgagcccaggagggttgaggctgccacgagcaaggatcgtgccactgcactccagcctaggcgacagaacaagaccttgtctcaaaagaaaaaagaacttttttttttaagttacctgtagtgcccagcccaagcaggtgctgagccagacttcattcctatcattgtccttattacgcagtgacttccccctcctcatttctctccactctgccacgcacacaccctcaccctccagcccataccaaccaccccaaccactgcctgtggtttcccatgtgcacccaggccaggcattttcacggcctttcctcctgacctacgcctggctcagctttctaggcccaagttcaaagacacctccctaaatcttcccagatccctctgctactgcccagcaccaccatcttatcacagccccacgtcgttcccaagtgctctccgatttctgcttaactccatgcctctcgctgtgtgtccgcatctcatcaataagtcctcaagtcctcttccatcctgctagcttcctcatcgctcgggaatcatccccgctacttcctggggaaactgactcccttctgggcacacacagtgctacccccggggaaatctaagaagagacccaggagaagataagcacggagagtcagagaatcaaggggaaagaaagggagagaggccgggcacagtggctcacacctgtaatccagcactttgggaggccaaggtgggtggatcacctgaggtcaggagtttgagaccagcctggccaacatggtgaaacctcttccctactaaaaatacaaaaaacatttagccgggcgtggtggtgggtgcctgtaatcccagctacttgggaagctgaggcaggagaactgcttgaactcaggaggcggaggttgcagtgaactgagatcacaccactgcactccagcctgagtgacagagcaagactccgtcaaaaaaaaagaaagaaagaaagaaaagaaggaaggaaagaaagaaggaaggaaggaagggaggaagggagagaggaagggagagaggaagggagagagagaaaaaaagagggagagagacacaaatacagagactgagatgggagagagagagagatggaagctccctcccctccatggccagggagacagatggagcaagagacctcaggggtgggcagacttggaggagaaggaccaggaggatgtggagtgccgaaatctccagtcagggccaggtgggcagtcagagactgcaaaggaggactgtcagacagggacaaaaggaagccattgatgtaaccgccctcccgcctgcccgccggaagagaggttgaggccggagctgctgggagcatggcactggggtgctgggaggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcaggtgtgtgagtcaccccaaccgcactgaacctgggcaggctgcttcccagtgccggagggctctagagcccggagtgagggcctgcaggtccctgggtggcacagagagtgctgggggtgcagggaggcctggggcaccatctgcttgccccagaggccggaatttgtcttcagacactttctttctccaaaacccggaggtctaaggactgagccgactagaacttcctctgcctcagattcaggccccagcccctcctccctcagacccaggagtttaggtcctagcccctcctccctcagacccaggagtccaagttcccacctcctccctcagactcaggagtccaggcccccagcccctcctccctcagacccaggagtccaagttctcacctcctccctcagacccaggagtccaggccccaagcccctcctccctcagacgcaagggtccaggcccccagcccctcctccctcagactcaggagtccaggcccccaagcccctcctccctcagacccaggagtccaggccctcactgcactcagggaccagtgctcccttccctggaggcctggtcaggggtcaccaagagcagagcgtgggggcgggaggaatgtgtgtgggaggcctgggtaaggaggaaaagggtgtagccagtctcctggctcagggacctgagagacaggggttaaaaggacgttccagaagcatctggggacagaaccagcctcttccagggaggcctgggagctgggggtgtgtgtctggcagtccctgcagccctgggctctgcggcccctgcgtcctccgcttggctctgccactgcatctgagtgtcttctctcctcacggctccccgcatttctaactctttctgcctcctcgtctcaaagctgttccttcccccgactcaagaatccccggaggcccggaggcctgcagcaggtgagatcacagacatcacagaacctgccgggtgggcggggtgggtggccattgcgcacagagccaggctccgaggaaaactcccatacagaggaagaacgctagggccccctatggtaaccctctcctgtcgacaggaaggcaaatcagtgcccaagaaagtagaaagatctaatcagaatctcaccatgggttactggaccagtggacgtagttgaattctctttggcactgttttcgtggatcctcttggaagatgtgggctgaggaagaataaatcaggaggctagatgggaaggacagaggtcaaggcaggagaccatagcaggccaggaaggaaggagaggatgcagagggagcagacagagggatggggggagggtcgaggcagtgactaatggaccatgtggcttcccctctcaggagcggccatgaagaagctgatggtggtgctgagtctgattgctgcaggtggggaaagggcatttggatgggggaggcttgcagacagggttgggcttgttgatggagaagaggctggtattggggatggggatatgcacagggttggggtgggggagctttgaaatgaggaagacgttggggattaggctaagggtggggaatacagatagggagggtggtgggaggtgggtttgaagatatgagggtttggggtggggttggctttagggatggggatctaaacatagaagaggtaggaggtaggttggaaagttggagagagcccgggaataggggatacagttgggtttgtaatgggaatggggtaagtttgggagtggaaatacagagaagctttttttttttgagacagggtctcactctgtcacccaggctggagtgtagtggcatgatccatagttcactgcagacttgaactcttgggtctcaagtgaccctcccacctcagcctcccaagtagctgggactacaggcgtatgccaccataccctgctaatttgtgtgtgtgtgtgtgtgtgtgtgtgtgtgtgtgtgtgtggagatgaggtctcactgtgttaccgaggctggtctcaaactcctgggctcaagcgatcctcctgcctcagctgggattacaggcataagccactgcacctgaccaatcttgactggagttcatgttgagggggatgcgcttggtttctccagaactcctctctgactcagatcttctctccctcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgtgagtctacactgtaaatgaacagcagatgcgactgaaccctgagggtgtcttatagatgtcaggcaggaggtgacataggcatcccccccatcccagcacgaggccatctgatagccaggtgcattcggctgttgcttaattgagtacttaatgtgtgccaggccctgcgggcatagcagtggaaaagaaaataaaaaaaagaaaacaaaaaaaaacaagcaaaattgctgttttcctgaacttactttctaatgggggaattggatcatttggggacctgcagggcgtgatgggcatttggatttaattctgagcacagtaggaagccactgggcagttttgtttttgttgtttgtttgttttttgagacacagtctcgctctgtcacccaggctggagtgtagtggcatgatctcagctcactgcaacctctgcctcccaggttccagcgattctcctgcctcagcaccccaagtagctgagattacaggtgtgcaccaccttgcctggctaatttttgtatgtttggtagagacggggtttcaccatgttggccaggctggtctcgaactcctgacctcaggtgatccgcccgcctcgccctcccaaagagctgggattacaggcatgagccaccaccacacccagcctgatttacatttttacaagcaccctggctaccacgtggaacgtggtctgggcaagagagagggagggaggcccacgtgggggctgttgctttcatccggcgacataggagggtggcttgaacccaggcggtcgcagtggggatggagggatgttgaatatcttgggatgtggaattctgagactgagccagcagaatctggcaacgaggaacaggagggagaggaagaagcacggctggcttccgtgtatttgtcctgaacaactgggtgttttgccacgtctttctctgagttgtgggagagggaaagagaaacaggccgggtgtaggcaggggagcatctgacattttgctttagccacgatgagttggagatgccggggagatgtcccagcagggaggccagggaggactctggagctcagaggagaggtcagggctggaggttaaaatgaaggcatcgtcagcaaacaggtgtatttaaagccatgggactagatgagatcatccaaaaagctggcatagttggaggagctggagggcccaggacaaaaaccctgggcgctgatcctcactagtcagattcacgacagctgccacttgtttgatgctaactaccaatcaggtgctgagtgaaaccatgtacacacctttcctggaatgcccaccacaagggactcttggcaccattttgcaaatgaggaaactgaggtgcagggaaatagcaagtgacaatccctggggtggttcccctgaccccaaggagaccttggatgactctcaccaccatcattcattcctttgatgtacattgactaagagcacctgctaagtgccacattcgagttgggcagtggagattcagcaatggatgggacacacacgtcatccctgccctcgggagcacaaggacagaaaggtgcagacaagcaaagtgagggctgggcatggtggctcacgcctgtaatcccagcactttgggaggccgaggtgggtggattacctgagttcgagaccagcttggccaacatggctcaaccctgtctctactgaaaatacaaaaaattagccaggcgtggtggtgggcttctgtaattccagcaacttgggaggctaaggcaggagaattgcttgaacgtgggaggcggaggttgcagtgagccgagatcgcgccactgcactccagcctgaaccacagagcgagactctgtctaaaaaaaaaaaaaggaaagaaagaagcagcaaattgggctggccgtggtggctcatgcctgtaatcccagcactttgggaggccgaggcgggtggatcactcgagcccaggagtacaaagctgcagtgagctgtgatctacagaacaccactgcagatccagcctgggtgacagagcgagaccctgtctcaaaaaaacaaacaaacaaaagaagcaaacccttcaaaaccccatataattacaaattatgaaggaaaagaatacgggtacctactttagatggaggagggtcaggaaggactttctaatgagataaaatccaagcggaggcatgaagatgggaaaaggaatgttcagggcagaggaaaggctgtgataacacccctgaggtgagaaccgtcttgagtattctcagaaaataaaatttcccgttcactggggggcagaaggtgctgggagataaggttggaaagtgactacagccagatcacacaggggctccagtgccaagtggaggagcccaggctttattcttaggacaatggggagccatgggtgatgtctgagcaagggagtgactctctgtttcaggaatatgtatcaaacacctatcctgtgccaggtgctgatcaacgcactggagatactatatctgaatagaacaaaaatccccatcttgacatcctagagctgcactgtctaatatggtagccatcagccacatatagcaaattacattgaaattaatgaaatggaaaatccacaagccacatttcaagtactcagcagccacctgtagcttgtggttcccccagccacctctggacagtgcagatcgagatcatggcatcgtagcatttagtggacagcattgctctgcaaggaggagaaataacacaatgagtaaatatttaacaataaatatatagcaggtcggatgattgtgataggttctctggtggaacagaaagcaggggagggagataggaattgcctactaacaggtatttgtattttaattgggcaactaaggaaggcttccctgagaggcgacatttaaaggaagtgagggagtgagctatgcagatacttggaggacagacttgctggcagagggaacagcagtgcaaaggccctgaggtgggaagatcactattgtgttcaaggcaagacagggaagccagcgtttggctggagcagagggagagaaggggagagtgggaggagaagatgtctgtgagatgatggggcagtgcttgcaaggcctggtgtgccacgttgagaactttggctttgattctgagtgagatgggagtcataggaggggctgagcagaggaggcacaggaccaacttacattgttaaaatatctctggttgctttgtggaggatggactgtgggggaccagagacagagcagggagcccagtgaggaggctactgctctagttcaggtaggaagtgaaaaggcagctcaaaccaagatggtagccgtgggaaaggtgagatgtggccagattctggatatgcttcagagaggcaaaaggaattctggacagcttggatgtagggcatgaaataaagagagtgaagaatagcccccaagattattctgaaaggatggaattgccatttacccagctggggaagactgtgggaggagcaggccagcgattcatgacttcccagccctctctgaagcctcaactgcagcccaagggctccaggtgagacccagccctcttccttcccaggaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtcagtagtgggaggctggtggggagcaggctactggctacttggggaagtgtgccaaaggatggggagtgggaaaattggtgaggggccatgggaagatgggctaatggtgaggaccaatgggacagggtttcaatgggagaaaggtcaagggggagggagagtgaatttgggagctgggccagtgagtgaacagccaatggaaaatgtagaccaatgggtgaatagcatgggagagatggaacataagatgaaggttcaataaagagggaaggtcagtggggagatgctaatcaggaaggatgtcaaaggtcaaaggggactgatcaggattcattgaacagcaggaaggaataatggagaaggaactgatggaagaagagaaaccaataaagcacaaaagccaactgaaggatgtgaattgagacagtgaatgggggtatagctgatggaagagggactaaggggaaaggatcaatggtccagaggagtcactagaggaaaaaacaggtccaatagatcagcaggatccatgaaggtgggcctgtgtgtgaagggccaataagaaaggtgaaccattggatgaagggccagtgggaaggcagagacaatgggggaggatgcggcaagttagaaaaggaccaatgagggaggtggaccattggatgaagggctaataggaagggagagccagtgggggatggtgaggccagttagaaaaggaccaaggagggaagcagaccaataggaagagagagccaatgagggagggcagggccagttaggaaaggaccaatgaggaaggtagaccattggaggaagggccaatagaaagggaggatccatgagggagggtggggacagttagaaaaggaccaatgatggaggtggaccattggatgaagaaccaatagaaaggaagaaccaatgggagagggcatggccagttaggaaaagaccaatggtcacagagtgaccaatcaagatgaatcaatgggcaggaagtgtccaatgaagaatggactactgatcaggaggggtacagtagaggagggcgtaacagaggaagagtcctccaggtcaactgaaactactgaagaaggtgggaccagtggaagagagaaaagtggaggagggacctaagagaaaaggaaaaccaataggaaatgaggactcctggagaagagactattaatgaggaagacagccaatgggggggaagaatgatagaaagagggaccaattaggaggcagggacgatggtaatgagatgtaagaatgagagacaaacaggaagaggggtgccaatagaaaagagggaccaatagaggatggaggacttataggggttggggggtgactggggaggatgaggggagtgcaaggcctgggctgagtctggcccatctctcccctaacaggtgatttccctgacaccatccagtgtgcatacatccacctggtgtcccgtgaggagtgtgagcatgcctaccctggccagatcacccagaacatgttgtgtgctggggatgagaagtacgggaaggattcctgccaggtgaggtgacccggatctgccacttacacagccagggacaggacgaagtcacaaaaacatggccagacacaggaagagagagacacaggccaaaagagagctttacagagacagatagagacaggctgagggagaacccaagccttgaaaagaagagacttagttcaacacacagagacacagtcagggatatgcagagatataaagacacagccagcagagacaggaagtgcagagacaaggatggaggccgcgggatcaagaaccagagaggccaggagcagcggctcatgcctgtaatcccggcactttgggaggccgaagcaggaggatcacctagggtcaggagttcgagaccagcctgatcaacatggtgaaaccctatctctactaaaaatacaaaaattaggatgggcacagtggctcatgcctgtaatcccagcaccttgggaggccgaagcaggaggatcacctggggtcaggagttcgagaccagcctgatcaacatggtgaaaccctatctctactaaaaatacaaaaattaggatgggcacagtggctcatgcctgtaatcccagcaccttgggaggccgaagcaggaggatcacctggggtcaagagattgagaccagcctggccgatatggtgaaaccctatctctactaaaaatacaaaaattagctgggcctggtgcaggcgcctgtagtcccagctactcaggaggctgtggcaggagaatcacttgaacctggaggcggaggttgttgcagtgagtcgagatcatgctactgcactccagcctggcaacagagcaagattccgtctcaaaaaaaaaccaaaaaacaaaaattacgcaagcatggtgggacacacctgtagtcccagctactcgggaggctgaggctggagaattgcttaaacccaggaggcagaggctgcagtgagctgagatcacgccactgcactccagcctggggacagagccagactctgtctaaaaacaaaaagaaccaaagagaagtagtaaggaagcagatggtgtgaggggactgtccttcctcaaacagagcccccacgagtcctgctcagaaacgaccaggctctggaggagggagacactagctggggaaaggggactccctcccgaatactttaacttgggtttcctccattgtcatccatccaggctctcctctttatgccagaatgactaatgcactgagggatgtgcagagaccaaccaagggggagacacaggcagaaacggagacacaggcagaaacagggacagagacagggaaagcgatacatagcaagttggacgcaaagaaagggcaggtgggcgagactgtcctcaagacacgaggtggagaggtgtccctggacagaatagtgccaggcatatctctccctgggccctccctacctctcccacctgggtcttatcgtctcctcctccccctcctccctctcctcctcttcctcctcctcctcctccctcatcatcttcttcttttctctctctctccatcggtctctacacctctgcctctctccacacctctcagtctccattcttaaattgtttctctttcttgctctctatgttcctctgcatcttggcattcctatctctgtgtctttgagtctcctttattctctctctaccattctctctctgtgcctttgtgtgtcttactgtctctctctctgtctctctgtccctgagtctttctctccatctttcagtaagtacctctgtccctttctacctctctctctgtcacacacacacacacacacacacacacacacacacacacacacacagtctctgggtttctatctgtatctgactttctccctctttcctgcagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaatttt

SEQ ID NO: 7 - DNA, 1506 bp, Homo sapiens
  ref <308>: DATABASE ACCESSION NUMBER: GenBank / U62801
  ref <309>: DATABASE ENTRY DATE: 2001-05-15
  ref <313>: RELEVANT RESIDUES: (1)..(1506)
aggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccatcaggcagtggctgttggaaagatttaagatttcacacctatgacatacatgggatagcacctgggccgccatgcactcaataaagaatgtatttt

SEQ ID NO: 8 - protein, 276 aa, Homo sapiens
  ref <308>: DATABASE ACCESSION NUMBER: GenBank / AAC14266
  ref <309>: DATABASE ENTRY DATE: 2000-08-30
  ref <313>: RELEVANT RESIDUES: (1)..(276)
MRAPHLHLSAASGARALAKLLPLLMAQLWAAEAALLPQNDTRLDPEAYGAPCARGSQPWQVSLFNGLSFHCAGVLVDQSWVLTAAHCGNKPLWARVGDDHLLLLQGEQLRRTTRSVVHPKYHQGSGPILPRRTDEHDLMLLKLARPVVPGPRVRALQLPYRCAQPGDQCQVAGWGTTAARRVKYNKGLTCSSITILSPKECEVFYPGVVTNNMICAGLDRGQDPCQSDSGGPLVCDETLQGILSWGVYPCGSAQHPAVYTQICKYMSWINKVIRSN

SEQ ID NO: 9 - DNA, 1580 bp, Homo sapiens
  gene (1)..(1580)
  ref <308>: DATABASE ACCESSION NUMBER: GenBank / NM_002776
  ref <309>: DATABASE ENTRY DATE: 2003-12-21
  ref <313>: RELEVANT RESIDUES: (1)..(1580)
catcctgccacccctagccttgctggggacgtgaaccctctccccgcgcctgggaagccttcttggcaccgggacccggagaatccccacggaagccagttccaaaagggatgaaaagggggcgtttcgggcactgggagaagcctgtattccagggcccctcccagagcaggaatctgggacccaggagtgccagcctcacccacgcagatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggctccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgaccacctgctgcttcttcagggagagcagctccgccggaccactcgctctgttgtccatcccaagtaccaccagggctcaggccccatcctgccaaggcgaacggatgagcacgatctcatgttgctgaagctggccaggcccgtagtgctggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccctgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatggtgattgtcatgtaaggcttaacacagtgggtggtgagttctgactaaaggttacctgttgtcgtga

SEQ ID NO: 10 - DNA, 5574 bp, Homo sapiens
  gene (1)..(5574)
  promoter (1)..(47)
  mRNA (48)..(120)
  5′UTR (48)..(120)
  mRNA (605)..(701)
  5′UTR (605)..(613)
  mRNA (2455)..(2635)
  mRNA (3589)..(3863)
  mRNA (4195)..(4328)
  mRNA (4793)..(5474)
  3′UTR (4946)..(5474)
  ref <308>: DATABASE ACCESSION NUMBER: GenBank / AF055481
  ref <309>: DATABASE ENTRY DATE: 2000-08-30
  ref <313>: RELEVANT RESIDUES: (1)..(5574)
ttggggtcaaaagggaaggtcccgccaggggtccctgggcagaggataccagcggcagaccacaggcagggcagaggcacgtctgggtcccctccctccttcctatcggcgactcccaggtgaagctacctgcaccccacccgggttggggtggattgcgagaggatgggtgggaacccccgggccacaggcaggagccggcttagagcctcggtttctccactgcgggacgcggaagtccccccgctgtgaggttgagaaagaggctcccactggctccgagcctcggatccccaccccgcgcgtgaaggagggggaaacctcgggcgcggcgtggctgcagccagggtagctggggcgcggagagcgctccactcgggcacagggaggacgggaagatgccgcgaggggcgtcattagggtaattgtgcccattaccgtgttccagcccgaatctcccgtctgccagcccctggggttatcggctgcaggtcaaaagggtgcttgcgggctggggtggcccaggctgggcggatgagcgcggcgaggtgggggtctctaacggagcatctgttttaacccgcccctgcacacaccccagcagatcctggccatgagagctccgcacctccacctctccgccgcctctggcgcccgggctctggcgaagctgctgccgctgctgatggcgcaactctggggtaaggtgggggacagggggcggggagaggcgccggtgggaggcacgggcgggagggcaatgtgttcccgtcaccaagccccgcgcacctctcctcccccgagaccccagcacccacccagcgctccggagcacggcccgcccccaagtcagctgggcccttcttctggctcggcccctgggtgacccgccccactcaggccctgtccgatttctgccacccggatcctcgctcttccgtggactcttcggcgtgttctttctcctcctcttacgccgccccatcccggccccgctccattttacactaccgtgttttgttttgtttgtttgtttgtttgagacggagtctcgctctgtcgcccaggctggagtgcagtggcgcgatctcggctcactgcaagctccgcctcccgggttcacaccattctcctgcctcagcctcccgagtagctgggactacaggcgcccgccaccaagcccggctagttttttgtatttttagtagagatgggatttcaccgtggtctcgatctcctggcctcgtgatccgcctgcctcggcctcccaaagtgctgggattacaagcgtgagccaccgctcccggccacaccacagtgttttatcctgagtcttgccttaccgctttttgccctctcccctcactttttttcttcctctttccctttctctctcttttttctttttctttctttcttttctttctttcgttcttcttttccttccttcctttccttccttcctttcgtttgtttcttctttcttgttttactttctctcttattttttcttctttctttcttgttttttttctttctctctctttttctttccttctttttttttttttttttttgagacagggcagtgctctgtctccgtggctggagtacagtggcccaatcagagctcactgcagcctcgacctcctgggctcaagcgatactcagcctccagagtagctggtaccacaggcatgcaccaccacatccggctttttttttttttttttttttttttttttttgagacagggtctcactctgtcgcccagactggagtgcagtggcccaatctcggctcattgcaacctccacctcctgggctcaagcgatcctcccacctcagcctcccaggtagctgtgactacaggcgcatgcctccgcgactacttttttgttgttgttgttgtttgtttgtttttgtagagactgggtcttgctgtgttgcccgggctggtcttgaattcccgagctcaagcggtccaaccgcctcggcttcccaaagtgctgggattacaggcgtaaaccactgcgccccacccctctcctggttttcaatcccgttttgttattcacaccccttcctctccccgatccccgagttctatccccgcacccttacctccccgccgcgttcaatccccgcccctctatcgaccagcgacgttctagccagctctccaggcgcgctgcgttcagtccctgccctccagacccaccctattctgtctcattactccacgctaccctatcccagcttccttccactttcacgcgcttcttctcctcccattccttcggtgcacgcgaaacccccaatatttccctaccaccctcgcgttctggctgcgtcccccgtccccgaacgcagtccagtgccacagcccagctccaaccccaaacccagaccctgccctccgtgctttgattccgtcccctttctttctcccagccgcagaggcggcgctgctcccccaaaacgacacgcgcttggaccccgaagcctatggcgccccgtgcgcgcgcggctcgcagccctggcaggtctcgctcttcaacggcctctcgttccactgcgcgggtgtcctggtggaccagagttgggtgctgacggccgcgcactgcggaaacaagtaggaggagatccatccccgaggacgccacggggggctgtggaggcggcctccagggaggagcgggcagggcggggtctctcggaactcccacagctggaggtgcggtccccggtgtccttccagcaggaggagagaccgggcttgcgctgtggccgccaggggacggtgtggtccttttccgcatttctgggcccgtcactcctctcccgctgattctccttgagctctaggaggaggtggttgcctgcaacggatagaagccagggtccggtgtggtcaggaatttagaactaaaggaaaaggttcacttcgtgagtccccgttgaaggaggaagggttgggtattaccacagagaaaatgtggagttgggctgggctcggtggctcacgcctgtaatcccagcactttgggaggccaaggcgggtggatcacctgaggtcaggagttcaagaccagcctggccaacatggtgaaaccacttctcaactaaaaataaaataaaataaaaaataaaaaattttaaaaaaattaaaaaaaaaaaaaggctgggtgtggtggcgggtgcctgtaatcccagctactcaggaaggaggctgaggcaggagaactgcttgaacctgggaggtggaggttgcagtgagccgagattgcgccattgcattctagcctgggtgacaagagcgaaactctgtttcaacaaagaaaagaaaaagagaaaagaaaatgtggaaggcttacctaggtgtccaggcccccagccctcctcaattcctgcagatcctcagagctcaaacaactgattcctcctccccatgtccactgaggtccccttctcccacaaggccctcttccctcagactcttcctatctccaggccctgcttcactgcccacctgctttcccagtccctgtgaagggtttgccttcacatgcctcttccttcccccaggccactgtgggctcgagtaggggatgaccacctgctgcttcttcagggcgagcagctccgccggacgactcgctctgttgtccatcccaagtaccaccagggctcaggccccatcctgccaaggcgaacggatgagcacgatctcatgttgctaaagctggccaggcccgtagtgccggggccccgcgtccgggccctgcagcttccctaccgctgtgctcagcccggagaccagtgccaggttgctggctggggcaccacggccgcccggagaggcaagagctggggctctgaggccagaacctcaggaggagggggctgagggcctgaacccctgggtctgaggaaggatgggctggggactggattcctggatctgagggaggacgggctggggtcctagatgcctgggtctgtgagtctgaggggaggaggggctgggggcctggactcctgggtctaagtggggaggggctggggccaggattcttgagtctgaaggaggaggggctggggcttaggatagaaacggtcttgtatctggactcctggctccccaaggattgggggctggacccagggattactggcatattctcccttcagtgaagtacaacaagggcctgacctgctccagcatcactatcctgagccctaaagagtgtgaggtcttctaccctggcgtggtcaccaacaacatgatatgtgctggactggaccggggccaggacccttgccaggtagggtctgaacagggagagtctctgactcctgggagggaggacagggaggttatgggaaaagagcagaccctgtgcccgatcccaaactccattcccaaacccatccttgaccccaactcttacccagacctaaccccctcctcatccctatcctcaatcccatttccatcctaaccccaccccattcccatctccaagcccattttcatcccctcaccttccatgaactacaatcccaacccaagtctcactgtgccttcattctcatcccccagcccaacctcccataacctgaagtccacctccattcctaccttccagctcatacctaattccaaccccatcccatcctcgtctttatcccaacccaaccccttccttccccaccactgccccagatcccaaagtgacagctctcacgttggcacatttatttgatctctcctttctgccacccccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccccgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatgtgattgtcatgtaaggcttaacacagtgggtggtgagttctgactaaaggttacctgttgtcgtgatctgaccacgtcccggtgaaagcgtgtgtccagggaagaagtgcacagggtagcccccaatcccaaccttccatccccaacccttagggatgatggaaga

SEQ ID NO: 11 - protein, 282 aa, Homo sapiens
  ref <308>: DATABASE ACCESSION NUMBER: GenBank / BAA33404
  ref <309>: DATABASE ENTRY DATE: 2003-01-31
  ref <313>: RELEVANT RESIDUES: (1)..(282)
MQRLRWLRDWKSSGRGLTAAKEPGARSSPLQAMRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 12 - protein, 250 aa, Homo sapiens
  ref <308>: DATABASE ACCESSION NUMBER: GenBank / AAD47815
  ref <309>: DATABASE ENTRY DATE: 2000-04-26
  ref <313>: RELEVANT RESIDUES: (1)..(250)
MRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 13 - DNA, 1186 bp, Homo sapiens
  ref <308>: DATABASE ACCESSION NUMBER: GenBank / AB012917
  ref <309>: DATABASE ENTRY DATE: 2003-01-31
  ref <313>: RELEVANT RESIDUES: (1)..(1186)
aggaatctgcgctcgggttccgcagatgcagaggttgaggtggctgcgggactggaagtcatcgggcagaggtctcacagcagccaaggaacctggggcccgctcctcccccctccaggccatgaggattctgcagttaatcctgcttgctctggcaacagggcttgtagggggagagaccaggatcatcaaggggttcgagtgcaagcctcactcccagccctggcaggcagccctgttcgagaagacgcggctactctgtggggcgacgctcatcgcccccagatggctcctgacagcagcccactgcctcaagccccgctacatagttcacctggggcagcacaacctccagaaggaggagggctgtgagcagacccggacagccactgagtccttcccccaccccggcttcaacaacagcctccccaacaaagaccaccgcaatgacatcatgctggtgaagatggcatcgccagtctccatcacctgggctgtgcgacccctcaccctctcctcacgctgtgtcactgctggcaccagctgcctcatttccggctggggcagcacgtccagcccccagttacgcctgcctcacaccttgcgatgcgccaacatcaccatcattgagcaccagaagtgtgagaacgcctaccccggcaacatcacagacaccatggtgtgtgccagcgtgcaggaagggggcaaggactcctgccagggtgactccgggggccctctggtctgtaaccagtctcttcaaggcattatctcctggggccaggatccgtgtgcgatcacccgaaagcctggtgtctacacgaaagtctgcaaatatgtggactggatccaggagacgatgaagaacaattagactggacccacccaccacagcccatcaccctccatttccacttggtgtttggttcctgttcactctgttaataagaaaccctaagccaagaccctctgcgaacattctttgggcctcctggactacaggagatgctgtcacttaataatcaacctggggttcgaaatcagtgagacctggattcaaattctgccttgaaatattgtgactctgggaatgacaacacctggtttgttctctgttgtatccccagccccaaagacagctcctggccatatatcaaggtttcaataaatatttgctaaatgagtg

SEQ ID NO: 14 - DNA, 9120 bp, Homo sapiens
  gene (2313)..(7622)
  mRNA (2313)..(2398)
  mRNA (4189)..(4263)
  mRNA (5061)..(5217)
  mRNA (5545)..(5810)
  mRNA (6627)..(6763)
  mRNA (7158)..(7622)
  ref <308>: DATABASE ACCESSION NUMBER: GenBank / AF164623
  ref <309>: DATABASE ENTRY DATE: 2000-06-26
  ref <313>: RELEVANT RESIDUES: (1)..(9120)
tgataatagtgttctctctcctcattggtcagggccccagccattgtccttgagagaatgctcgactctttatgttgtcttgacagcctcccctgagattggtcattaatgactgtgctctctctcctcattggtcagggccccagccattgtccttgagagaacctctgtcctttatggagttccacccttcttccctgggattggcccctagagacagtggttcttctcttttggttagccattgccattgtcctccgggaaagtgattatactcttttgtctaatgaccagacttggagccctccccaaggcccaggactgggttgaagggttggggaggaaaacagaaataagatgtctcccttgttcagacagtacttctcttcccttccagggtgattctgggggccccctggtgtgtgggggagtccttcaaggtctggtgtcctgggggtctgtggggccctgtggacaagatggcatccctggagtctacacctatatttgcaagtatgtggactggatccggatgatcatgaggaacaactgacctgtttcctccacctccacccccaccccttaacttgggtacccctctggccctcagagcaccaatatctcctccatcacttcccctagctccactcttgttggcctgggaacttcttggaactttaactcctgccagcccttctaagacccacgagcggggtgagagaagtgtgcaatagtctggaataaatataaatgaaggaggggccatgtctgtccatttgaagtcctcatgctggttgagactggaagaaggactcagcagtttccctatctcataggagtagaaacagagctcaaataaggccaggcacagtggctcacacctgtaatcccatcactttgggaagctgaggcaggtggatcacctgaggtcaggaactcgggaccagcctggtcaacatagtgaaaccccaactctactaaaaatgcaaaaattagccaggcatggtggcgcatgcctgtaatcccagctactcaggaggctgagacaggagaatagcatgaacccgtgaggcagaggctgcagcgagccgagattgaaccattacactccagcctgggcgacagagcgagactccatctcaaaaacaaacaaacaaaaaacccagtgctcaaataggatgagggtcttccctgagtagttactcagaaatggagtagaaaaagttacttttaataatataggccgggtgcagtggcccacgcctgtaatcccagcactttgggaggccgaggtgggaggatggcttgagctcagatttcgagatcagcctggcaacacagtgaaatcttgtcactacaaaaacacaaaaaattagctgggtgtggtggtgcgtgcctgtagtcccagctacttgggaagctgaggtgggaggatcacccgagccggggaggtggaggctgcaaagagccgagatcatgccactgcactccagcctgggcaataaagtgagaccttgtctcaaaaacaaaaacccagcaatataaataagacacatgtttcttcatctggcataatagaaatagtgcccagagcttataagcttttcaagagtccacaaaagacccgaaaaagaaaaagaaaattgttagctccaaaataccagatgaaagctgcaaagtcaacatttatgaccatttaatccaatgtccataaaacgtagcattctttccactagccaactgcagtttactttcttgtaatgaagcatacattgtatctttaatgtgggacgtggctttgttctaataagacgaagggtggagtgcaggcttggaaagcaggagagctcagcctacgtctttaatcctcctgcccaccccttggattctgtctccactgggactcaagaggtgaggagagaccatctccccaaatgcactgaagggaaactggaggagggagggagtgaggggtgatcataccagcggaggcacatttgctgagcccccccgcagtctgctctttccaagtggaccctcctggaagcctgatcccaacctcccctgcaagcaggtctgtcacccccatctctcagatgaagaaactgagccttgcaggggtggagtcccttgtccccacgtcataagggtagtcatagtagtaggaagaggaagcacctaggtttgaggccagggctggctgctgtcagaacctaggccctcccctgccttgctccacacctggtcaggggagagaggggaggaaagccaagggaagggacctaactgaaaacaaacaagctgggagaagcaggaatctgcgctcgggttccgcagatgcagaggttgaggtggctgcgggactggaagtcatcgggcagaggtctcacagcagccagtaagtgaacagctggactcgggctgcctgggcggcagggagaagcgggcaggggaagggtcagcagaggagcgaggccccagaggagccctggggtggagcacagccaagggctctgttccctttcctggactcggcttccacaggccctgacctgcctcccccaccctccggtcctgcccctgtgcctggcagcagccccacctgtgtgacatcccagcacaccccccctctccttgcaaaggagaagggagcggcctaggggaggccaggggcccacctgggctggggctgtggagagggagtggctgggacgggaggaaaaagagagacggagattagatggaagaagagggatttcaagacaaattgccagagatgcagtcagagagactgactgagagacacaaagatagaaggaattagagaaagggccacacagagccagacagagagagaagagtggagatggagacagggacgaggacagagaaaggcagacagacacatagggacagaaagagaaaaatcacacaaagtcagaattactgaatgacagggaatgacacatagaacgagacacagattcagagactcagggcagggaaaggaaggctgcagacagacagacagacagagggaggctgagacacagggagaagaggggcttggagaggtggcacaggcaggcagccagtgcctcagaggcctccggggagggccctcacacacaccccgccccggggcattaaggcagggcttggaggccagtcatcctgggcccgcccagggccgcccccctgccagcccgcctgcctggtgcctggcacctggcgctccaacccagcctacctgctgtagctgccgccactgccgtctccgccgccactgggcccccagagccccagccccagagcctgtgagtccaggaggaaagggaagctgcccctccccgtccaggtgtcagccctccccaaggacacctgtcccactcgggcacccatttctccctctgctctgtccttctctgcttgggtgggggttcctggcctctctctacacctctcacctccgatggctgtccgcagcctcagttacctctaatctccatggcttcagctgctgagctggccctctgctcccacccccgctggccagggcagcggagggcactggccctcccctcgaccagccccgcccagctttgcttggctgtccttcaaaagggcaggggtttggcggacagggcttcagcaagccgggtgatgggggtcccagacattgtctggggctgagccccctactcccctccagcagacctcaaaggctccatatcgctctgctgcgaagacaatgaaaaaggggtggctacggaacggtgtctggttccccttgtccttccaccccaagctgctggggcctggccagctctcaaggcaagaaggaaaacatcctctgacatgtgccggggaggtcccatggctgacttgaacagggccgaaccatggcttgacagctcaaagcccctcccaacgacttccacatggttcttggtatctcggaagcttctagctgtgaccaggccctctccaaggccaccctagacacctaagatatattttaagtgtttggagatctgagtgctgtgagaaacaggggatttccccaaccttgtttctcccaagtggggagcgggagcaggtgagggagagaggagagggcatgagccagcccccccctcccgatttccccgtaaagtgatgcggccccatgtccctccttgttcccagaggaacctggggcccgctcctcccccctccaggccatgaggattctgcagttaatcctgcttgctctggcaacaggtacgcaggggatgggggcagggcaggatcctccctcttgaatctctgggatcccctaaccctctgtgtctggacagtgacagggctgattccaaattacagaacaacccataaggcacctgaactggagcagtggtcatgagggcctggatgcccttctagataatccctttaaatgccaaaggaggagaggtcaagggggtcgtaaagggtcccgtggaggggctgaggaagctggagttgggggagcagtcactcaaagcgcccaggacaggggctactgaccaaccagtatggaagtatttccttttttttttttcccagagacaaagtcttgctctattgtccaggctggagtgccgtggtgccaacacggctcactgcagtcttgacttcccgggcttaagtgatccttaagccatctcagcttccccggtagctgggaccacaggcacctgccaccaagccaggctaattgtttaattgtttgtagagatgggggaggaggtctcactatgtttgcctaggctgatctagaactcctgggctcaagtaatcctcccaccttagcctctcaaagtgctgggattacaggcatgagccactgcatttgaccttatggaagtattttcatcctttaatacccgaccccagcatccagggcaacccagagggacaccagaccagggcccagaccacccactctctttctctcctccccacccccatttctgggagtcctcctggtctaccacctctccttcctgagccccttcttttgctctcaccccctccagggcttgtagggggagagaccaggatcatcaaggggttcgagtgcaagcctcactcccagccctggcaggcagccctgttcgagaagacgcggctactctgtggggcgacgctcatcgcccccagatggctcctgacagcagcccactgcctcaagccgtgggtgcgggggctggggcggtgccggggtggggggctgggaatggggagatggatggagagaagctcagggataggggtgctggtaaggggattagagatggggatgggtagtgtcagcaaggttgatgggctcgagttggtattgaaggtggggggatgaatggggttgggatggggctatggctgggaagggggcttcggtgggagacgtggaagaggttggaagcagagcgatgtttcttcatcctcaaaggtgtcactcacctctcccacccatgtctcccccgacctttcctcctccaactactgtctctcccacctcagccgctacatagttcacctggggcagcacaacctccagaaggaggagggctgtgagcagacccggacagccactgagtccttcccccaccccggcttcaacaacagcctccccaacaaagaccaccgcaatgacatcatgctggtgaagatggcatcgccagtctccatcacctgggctgtgcgacccctcaccctctcctcacgctgtgtcactgctggcaccagctgcctcatttccggctggggcagcacgtccagcccccagtgtaggagcaccagaggggaacctggcagggggtggtgaggagggagtggtcaggattgtggaagggttcagggcatcagagatgcggttcacagtgacgatgtgggataagttgagaggatgtgtggaaaacgtcaggataggggggtggggacaaaagttggggccttggagtcagacggacgggatatgcaatcatacatccataacctcctggttgtaagaccttaggcaagcagcttcacctctctgaatcttgattttcttctctataaaatgagaatgattatacccacctgtcaggattggattagagataatgtatatcaagcaactgacataaatcatttattggatagcaggctgggcaccgtggctcacgcctgtaatcccagcactttgggaggccgaggtgggaagatcacctgaggtcaggactttgataccagcctggccaacgtggtgaaatcccatctctactaaaaatgtgaaaattagttgggcgtggttgtgtgcgcctgtaatcccagctactcgggaggttgaggcaggagaatcgcttaaacttgggagacggaggttgcagtgagccaagatcacgccactgcactccagcctgggcaacagagcaagactctgtctcgaaaaaaaaaaaaaaaaagctggatagcattgctgttgctattgttacaagaagagaggtgagttggctgcgtctaaggacagggattcccccaggggcgggatcacagcaagcactgcattaggggaggtggcagggggctcattcccacagcccctcacgctgtttccacagtacgcctgcctcacaccttgcgatgcgccaacatcaccatcattgagcaccagaagtgtgagaacgcctaccccggcaacatcacagacaccatggtgtgtgccagcgtgcaggaagggggcaaggactcctgccaggtcagtgtggtctccaaccacagccccatccccatccccagcttcaatgacatctttaccgacatccacaatttcatccccaacctcaacccgccgacccctgcaactcccaatccatctcttcccctgttcccgtttctgacctcagcacaaacttcagctccatccccgtttccacaccatttccagctccaaccatccccaaactcgtttttgagcctaaccccatcctttatcccacccataatcccagctttatcgctaaacctatcacctttcccagtgcctacccatcctgtctcggccccactcctaagcaccgtccccacctcctccctggctaacaccatgctcaacgctttctctgaccgacattctctctccccgtgcccagggtgactccgggggccctctggtctgtaaccagtctcttcaaggcattatctcctggggccaggatccgtgtgcgatcacccgaaagcctggtgtctacacgaaagtctgcaaatatgtggactggatccaggagacgatgaagaacaattagactggacccacccaccacagcccatcaccctccatttccacttggtgtttggttcctgttcactctgttaataagaaaccctaagccaagaccctctacgaacattctttgggcctcctggactacaggagatgctgtcacttaataatcaacctggggttcgaaatcagtgagacctggattcaaattctgccttgaaatattgtgactctgggaatgacaacacctggtttgttctctgttgtatccccagccccaaagacagctcctggccatatatcaaggtttcaataaatatttgctaaatgagtgaatctactgagtgcttactatgtgctagaccctgatccaatggcttttattttattttattttttgacagagtctcgctctgtcacccaggctggagtacagtggtgctatctctgctcactgcaacctccacctcctgggttcaagcaattctcctgcctcagcctcctgaatagctgggattacaggtgcctaccaccacatccggctaatttttgtattttttagtagagatggggcttcaccatgttggccaggctggtctcgaactcctgacctcagatgatctgccctccttggcctcccaaactcctgggattacagacgtgagccaccgcgcccgcccggctttcatttattaattaaaagaaattaaattaattaatctatttaggagacagtcttgctctgttgcccaggctggagtgcagtaacaatcacagctcacggcaatctcaatttcctggggtcaagtgattgtcctccctcagcctccagagtagctgggactacaggcacatgccacgaagcccagctaatttttgtatttttcgtagagacagaggtctcagtatgttgccccggctagtctcaaactcctgggctcaagcagtctgtcctcctcagcctccaaaagtggtgagattacaggcatgagtcgctgtgcctggcctccaagcactttcaaatgtatcaacttaatcctcacaaaaccctgtgaggtcggtactgttttcatacctattttatagttgaagaaacagacacagagaagcaaagtcacttgctcacagtcacgtggctaggagagcaaggatctgaagcaaggcgatctcttaattaccaagtgatgttcctggagtaaggctctgtttgtttcctttcctgtaaaatgctgcatgcaaaagtataacacagtaagtaaagaagtcagttagcctgcacatactaagacctaaccaaaggagcttattgtttttctccaacttccatgataggtaattagatagtggagacctctgctggccaatatggtagccactaaccgcagctggctcttccaattaaaattacataaagccagaaatgtaactcctctgtctcacttgttatatctccaaggctggatagccacatgtgactggtggtggctggattagctagtgcatataaaacatcactgcagaaagttcagctgagcagcactgagttagatggcctctgaagaggatgtcccacggagagaatccagaactcaggatctttttttttttttttctttgcgacagagtcttgctctgtcacccaggctggagtgcagtggcgtgatctcggctcactgcaacttctgcctcccaggttcaagcaattctcctgcctcagcctccctagtagctgggactacaggcctgtgccaacatccccagctaatttttgtgtctttttagtagagatggggtttcactatgttggccaggctggtctcgaactcctgacc

(19) United States
(12) Patent Application Publication
(10) Pub. No.: US 2006/0141471 A1
(43) Pub. Date: Jun. 29, 2006

(54) Assay for detection of renal cell carcinoma

(75) Inventors: Eleftherios Diamandis, Toronto (CA); Constantina Petraki, Athens (GR)

Correspondence Address: MERCHANT & GOULD PC, P.O. BOX 2903, MINNEAPOLIS, MN, 55402-0903, US

(73) Assignee: Mount Sinai Hospital, Toronto, Ontario (CA)

(21) Appl. No.: 10/546,435

(22) PCT Filed: Feb. 26, 2004
(86) PCT No.: PCT/CA04/00280

§ 371 Date: Jan. 26, 2006

Related U.S. Application Data
(60) Provisional application No. 60/451,382, filed on Feb. 27, 2003.

(51) Int. Cl. C12Q 1/68; G01N 33/574

(52) U.S. Cl. 435/6; 435/7.23

(57) ABSTRACT

Methods for diagnosing and monitoring renal cell carcinoma in a subject comprising detecting kallikrein polypeptides, or polynucleotides encoding the polypeptides in a sample from the subject. The kallikrein polypeptides include kallikrein 5, kallirekin 6, kallikrein 10, and kallikrein 11.

### FIELD OF THE INVENTION

The invention relates to compositions, kits, and methods for detecting, characterizing, preventing, and treating renal cell carcinoma.

### BACKGROUND OF THE INVENTION

Kallikreins are a subgroup of secreted serine proteases, encoded by highly conserved and tightly clustered multigene families in humans, rats and mice. The human kallikrein gene family resides on chromosome 19q13.4 and is comprised of 15 members, whose genes are designated as KLK1 to KLK15 and the corresponding proteins as hK1 to hK15 (Yousef G M, Diamandis E P., Endocr Rev. 2001; 22:184-2041; Yousef G M, Chang A, Scorilas A, et al., Biochem Biophys Res Commun. 2000; 276:125-133; Diamandis E P, Yousef G M, Clements J, et al. Clin Chem. 2000; 46:1855-1858). Kallikreins are expressed in a wide variety of tissues and are found in many biological fluids (e.g. cerebrospinal fluid, serum, seminal plasma, milk, etc.) where they are predicted to process specific substrates. Kallikreins may participate in cascade reactions similar to those involved in digestion, fibrinolysis, coagulation, wound healing and apoptosis (Yousef G M, Diamandis E P., Endocr Rev. 2001; 22:184-2041). Many kallikreins have been found to be differentially expressed in endocrine-related malignancies (Diamandis E P, Yousef G M, Expert Rev. Mol. Diagn. 2001; 1:182-190), including prostate (Barry M J. Clinical practice, N Engl J Med. 2001; 344:1373-1377; Rittenhouse H G, Finlay J A, Mikolajczyk S D, et al., Crit Rev Clin Lab Sci. 1998; 35:275-368; and Yousef G M, Scorilas A, Jung K, et al., J Biol Chem. 2001; 276:53-61), ovarian (Kim H, Scorilas A, Katsaros D, et al., Br J Cancer, 2001; 84:643-650; Anisowicz A, Sotiropoulou G, Stenman, et al., Mol Med. 1996; 2:624-636; Tanimoto H, Underwood L J, Shigemasa K, et al., Cancer. 1999; 86:2074-2082; Magklara A, Scorilas A, Katsaros D, et al., Clin Cancer Res. 2001; 7:806-811; Yousef G M, Kyriakopoulou L G, Scorilas A, et al., Cancer Res. 2001; 61:7811-7818; Luo L, Bunting P, Scorilas A, Diamandis E P., Clin Chim Acta. 2001; 306:111-118), breast (Yousef G M, Magklara A, Chang A, et al., Cancer Res. 2001; 61:3425-3431; Yousef G M, Chang A, Diamandis E P; J Biol Chem. 2000; 275:11891-11898; and Yousef G M, Magklara A, Diamandis E P, Genomics. 2000; 69:331-341), and testicular cancer (Luo L Y, Rajpert-De Meyts E R, Jung K, et al., 2001; 85:220-224). In addition, many kallikrein genes examined thus far are under steroid hormone regulation, implicating a role for kallikreins in endrocrine-related tissues (Yousef G M, Diamandis E P., Endocr Rev., 2001; 22:184-204). Furthermore, hK6, hK10 and hK11 have been recently identified as novel serological ovarian cancer biomarkers (Luo L, Bunting P, Scorilas A, Diamandis E P., Clin Chim Acta. 2001; 306:111-118 Diamandis E P, Yousef G M, Soosaipillai A R, Bunting P., Clin Biochem. 2000; 33:579-583, and Diamandis E P, Okui A, Mitsui S, et al., Cancer Res. 2002; 62:295-300).

### SUMMARY OF THE INVENTION

The present invention seeks to overcome the drawbacks inherent in the prior art and provide sensitive and accurate methods for the detection of renal cell carcinoma. It has been found that one or more kallikrein polypeptides wherein the kallikrein polypeptides are kallikrein 5, kallikrein 6, kallikrein 10, and kallikrein 11, and polynucleotides encoding the polypeptides, have particular application in the detection of renal cell carcinoma. Thus, the kallikrein markers constitute biomarkers for the diagnosis, monitoring, progression, treatment, and prognosis of renal cell carcinoma, and they may be used as biomarkers before surgery or after relapse.

In accordance with the methods of the invention, the presence of levels of kallikrein markers in a sample can be assessed, for example by detecting the presence in the sample of (a) polypeptides or polypeptide fragments corresponding to the markers; (b) metabolites which are produced directly or indirectly by polypeptides corresponding to the markers; (c) transcribed nucleic acids or fragments thereof having at least a portion with which the markers are substantially identical; and/or (c) transcribed nucleic acids or fragments thereof, wherein the nucleic acids hybridize with the markers.

In an embodiment, the invention provides a method for detecting one or more kallikrein polypeptides wherein the kallikrein polypeptides are kallikrein 5, kallikrein 6, kallikrein 10, and kallikrein 11 associated with renal cell carcinoma in a patient comprising:
(a) obtaining a sample from a patient;
(b) detecting or identifying in the sample one or more kallikrein polypeptides wherein the kallikrein polypeptides comprise kallikrein 5, kallikrein 6, kallikrein 10, and kallikrein 11 associated with renal cell carcinoma; and
(c) comparing the detected amounts with amounts detected for a standard.

The term “detect” or “detecting” includes assaying, assessing, imaging or otherwise establishing the presence or absence of the target kallikrein polypeptides or polynucleotides encoding the polypeptides, subunits thereof, or combinations of reagent bound targets, and the like, or assaying for, imaging, ascertaining, establishing, or otherwise determining one or more factual characteristics of renal cell carcinoma, metastasis, stage, or similar conditions. The term encompasses diagnostic, prognostic, and monitoring applications. The kallikrein markers can be detected individually, sequentially, or simultaneously.

According to a method involving one or more kallikrein polypeptides the levels in the sample of one or more kallikrein polypeptides wherein the kallikrein polypeptides comprise kallikrein 5, kallikrein 6, kallikrein 10, and kallikrein 11 associated with renal cell carcinoma, are compared with the normal levels of the kallikrein polypeptides, in samples of the same type obtained from controls (e.g. samples from individuals not afflicted with renal cell carcinoma). Significantly different levels in the sample of the kallikrein polypeptides relative to the normal levels in a control is indicative of renal cell carcinoma.

In an embodiment, the invention provides a method for diagnosing and monitoring renal cell carcinoma in a subject comprising detecting in a sample from the subject one or more kallikrein polypeptides wherein the kallikrein polypeptides comprise or are selected from the group consisting of kallikrein 5, kallikrein 6, kallikrein 10, and kallikrein 11 associated with renal cell carcinoma. The kallikrein markers can be detected using antibodies that bind to the kallikrein markers or parts thereof.

Thus, the invention provides a method of assessing whether a patient is afflicted with or has a pre-disposition for renal cell carcinoma, the method comprising comparing:
(a) levels of one or more kallikrein polypeptides in a sample from the patient wherein the kallikrein polypeptides comprise kallikrein 5, kallikrein 6, kallikrein 10, and kallikrein 11 associated with renal cell carcinoma; and
(b) normal levels of the kallikrein polypeptides, in samples of the same type obtained from control patients not afflicted with renal cell carcinoma, wherein significantly different levels of the kallikrein polypeptides, relative to the corresponding normal levels of the kallikrein polypeptides, is an indication that the patient is afflicted with renal cell carcinoma.

In an embodiment of a method of assessing whether a patient is afflicted with renal cell carcinoma (e.g. screening, detection of a recurrence, reflex testing), the method comprises comparing:
(a) levels of one or more, preferably 2 or more, kallikrein polypeptides in a sample from the patient, wherein the kallikrein polypeptides comprise kallikrein 5, kallikrein 6, kallikrein 10, and kallikrein 11 associated with renal cell carcinoma; and
(b) normal levels of the kallikrein polypeptides, in a control non-renal cell carcinoma sample.

A significant difference between the levels of the kallikrein polypeptides in the patient sample and the normal levels is an indication that the patient is afflicted with renal cell carcinoma. In an embodiment, the levels of kallikrein markers are significantly higher and are indicative of tumors of high grade, advanced stage disease, and/or lower survival.

The invention further relates to a method of assessing the efficacy of a therapy for inhibiting renal cell carcinoma in a patient. This method comprises comparing:
(a) levels of one or more kallikrein polypeptides in a sample from the patient obtained from the patient prior to providing at least a portion of the therapy to the patient, wherein the kallikrein polypeptides comprise kallikrein 5, kallikrein 6; kallikrein 10, and kallikrein 11 associated with renal cell carcinoma; and
(b) levels of the kallikrein polypeptides in a second sample obtained from the patient following therapy.

A significant difference between the levels of the kallikrein polypeptides in the second sample, relative to the first sample, is an indication that the therapy is efficacious for inhibiting renal cell carcinoma.

The “therapy” may be any therapy for treating renal cell carcinoma including but not limited to chemotherapy, immunotherapy, gene therapy, radiation therapy, and surgical removal of tissue. Therefore, the method can be used to evaluate a patient before, during, and after therapy, for example, to evaluate the reduction in tumor burden.

In an aspect, the invention provides a method for monitoring the progression of renal cell carcinoma in a patient, the method comprising:
(a) detecting in a patient sample at a first time point, one or more kallikrein polypeptides in a sample from the patient wherein the kallikrein polypeptides comprise kallikrein 5, kallikrein 6, kallikrein 10, and kallikrein 11 associated with renal cell carcinoma; and
(b) repeating step (a) at a subsequent point in time; and
(c) comparing the levels detected in (a) and (b), and therefrom monitoring the progression of renal cell carcinoma in the patient.

In another aspect, the invention provides a method for assessing the aggressiveness or indolence of renal cell carcinoma (e.g. staging), the method comprising comparing:
(a) levels of one or more kallikrein polypeptides in a sample from the patient wherein the kallikrein polypeptides comprise kallikrein 5, kallikrein 6, kallikrein 10, and kallikrein 11 associated with renal cell carcinoma; and
(b) normal levels of the kallikrein polypeptides in a control sample.

A significant difference between the levels in the sample and the normal levels is an indication that the cancer is aggressive or indolent.

The invention provides a method for determining whether renal cell carcinoma has metastasized or is likely to metastasize in the future, the method comprising comparing:
(a) levels of one or more kallikrein polypeptides in a sample from the patient wherein the kallikrein polypeptides comprise kallikrein 5, kallikrein 6, kallikrein 10, and kallikrein 11 associated with renal cell carcinoma; and
(b) normal levels (or non-metastatic levels) of the kallikrein polypeptides in a control sample.

A significant difference between the levels in the patient sample and the normal levels is an indication that the cancer has metastasized or is likely to metastasize in the future. The invention also provides a method for assessing the potential efficacy of a test agent for inhibiting renal cell carcinoma in a patient, and a method of selecting an agent for inhibiting renal cell carcinoma in a patient.

The invention further provides a method of inhibiting renal cell carcinoma in a patient comprising:
(a) obtaining a sample comprising cancer cells from the patient;
(b) separately maintaining aliquots of the sample in the presence of a plurality of test agents;
(c) comparing levels of one or more kallikrein polypeptides, in each of the aliquots, wherein the kallikrein polypeptides comprise kallikrein 5, kallikrein 6, kallikrein 10, and kallikrein 11 associated with renal cell carcinoma;
(d) administering to the patient at least one of the test agents which alters the levels of the kallikrein polypeptides in the aliquot containing that test agent, relative to other test agents.

The invention also contemplates a method of assessing the renal cell carcinoma carcinogenic potential of a test compound comprising:
(a) maintaining separate aliquots of renal cell carcinoma cells in the presence and absence of the test compound; and
(b) comparing levels of kallikrein polypeptides in each of the aliquots, wherein the polypeptides comprise kallikrein 5, kallikrein 6, kallikrein 10, and kallikrein 11.

A significant difference between the levels of the kallikrein polypeptides in the aliquot maintained in the presence of (or exposed to) the test compound relative to the aliquot maintained in the absence of the test compound, indicates that the test compound possesses renal cell carcinoma carcinogenic potential.

In embodiments of the methods of the invention, two, three, or four kallikrein polypeptides are employed. In preferred embodiments, the kallikrein polypeptides comprise kallikrein 6; kallikrein 6 and kallikrein 10; kallikrein 6 and kallikrein 11; kallikrein 6, kallikrein 10, and kallikrein 11; or kallikrein 5, kallikrein 6, kallikrein 10, and kallikrein 11. In a particular embodiment, the kallikrein polypepetides are selected from the group consisting of kallikrein 6, kallikrein 10, and kallikrein 11.

Other methods of the invention employ one or more polynucleotides capable of hybridizing to polynucleotides encoding kallikrein polypeptides wherein the kallikrein polypeptides comprise kallikrein 5, kallikrein 6, kallikrein 10, and kallikrein 11 associated with renal cell carcinoma. Methods for detecting polynucleotides encoding kallikrein polypeptides can be used to monitor renal cell carcinoma by detecting the nucleic acids.

Thus, the present invention relates to a method for diagnosing and monitoring renal cell carcinoma in a sample from a subject comprising isolating nucleic acids, preferably mRNA, from the sample; and detecting polynucleotides encoding kallikrein polypeptides in the sample, wherein the kallikrein polypeptides comprise kallikrein 5, kallikrein 6, kallikrein 10, and kallikrein 11. The presence of different levels of polynucleotides encoding the kallikrein polypeptides, in the sample compared to a standard or control is indicative of disease, disease stage, and/or prognosis, e.g. progression-free and overall survival.

In an embodiment, the invention provides methods for determining the presence or absence of renal cell carcinoma in a subject comprising (a) contacting a sample obtained from the subject with oligonucleotides that hybridize to polynucleotides encoding kallikrein polypeptides wherein the kallikrein polypeptides comprise kallikrein 5, kallikrein 6, kallikrein 10, and kallikrein 11; and (b) detecting in the sample levels of nucleic acids that hybridize to the polynucleotides relative to a predetermined cut-off value, and therefrom determining the presence or absence of renal cell carcinoma in the subject. Within certain embodiments, mRNA is detected via polymerase chain reaction using, for example oligonucleotide primers that hybridize to polynucleotides encoding kallikrein polypeptides, or complements of such nucleic acids. Within other embodiments, the amount of mRNA is detected using a hybridization technique, employing oligonucleotide probes that hybridize to polynucleotides encoding kallikrein polypeptides, or complements of such nucleic acids.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of polynucleotides encoding kallikrein polypeptides in the sample. For mRNA the analyzing step may be accomplished using Northern Blot analysis to detect the presence of polynucleotides encoding kallikrein polypeptides. The analysis step may be further accomplished by quantitatively detecting the presence of polynucleotides encoding kallikrein polypeptides in the amplification product, and comparing the quantity of markers detected against a panel of expected values for the known presence or absence of the markers in normal and malignant tissue derived using similar primers.

In embodiments of the invention, one, two, three or four polynucleotides encoding kallikrein polypeptides are employed. In preferred embodiments, the polynucleotides encode kallikrein 5, kallikrein 6, kallikrein 10, and kallikrein 11; and kallikrein 6, kallikrein 10 and kallikrein 11.

The invention also provides a diagnostic composition comprising kallikrein polypeptides, or polynucleotides encoding the polypeptides, or agents that bind to the polypeptides or nucleic acids.

In an embodiment, the composition comprises probes that specifically hybridize to polynucleotides encoding kallikrein polypeptides, or fragments thereof. In another embodiment a composition is provided comprising specific primer pairs capable of amplifying polynucleotides encoding kallikrein polypeptides, using polymerase chain reaction methodologies. In a still further embodiment, the composition comprises agents that bind to kallikrein polypeptides (e.g. antibodies) or fragments thereof. Probes, primers, and agents can be labeled with detectable substances.

In an aspect the invention provides an in vivo method comprising administering to a subject agents that have been constructed to target kallikrein polypeptides.

The invention therefore contemplates an in vivo method comprising administering to a mammal imaging agents that carry labels for imaging and that bind to kallikrein polypeptides, and then imaging the mammal.

Still further the invention relates to therapeutic applications for renal cell carcinoma employing kallikrein polypeptides and polynucleotides encoding the polypeptides, and/or agents identified using methods of the invention.

The invention also includes kits for carrying out methods of the invention. In an embodiment, the kit is for assessing whether a patient is afflicted with renal cell carcinoma and it comprises reagents for assessing one or more kallikrein polypeptides, wherein the kallikrein polypeptides comprise or are selected from the group consisting of kallikrein 5, kallikrein 6, kallikrein 10, and kallikrein 11.

In another aspect the invention relates to a kit for assessing the suitability of each of a plurality of test compounds for inhibiting renal cell carcinoma in a patient. The kit comprises reagents for assessing kallikrein polypeptides, wherein the kallikrein polypeptides comprise or are selected from the group consisting of kallikrein 5, kallikrein 6, kallikrein 10, and kallikrein 11. The kit may also comprise a plurality of test agents or compounds.

The invention contemplates a kit for assessing the presence of renal cell carcinoma cells, wherein the kit comprises antibodies specific for selected markers, wherein the markers comprise or are selected from the group consisting of kallikrein 5, kallikrein 6, kallikrein 10, and kallikrein 11.

Additionally the invention provides a kit for assessing the renal cell carcinoma carcinogenic potential of a test compound. The kit comprises renal cell carcinoma cells and reagents for assessing kallikrein markers, wherein the markers comprise or are selected from the group consisting of kallikrein 5, kallikrein 6, kallikrein 10, and kallikrein 11.

In an aspect the invention provides a method of treating a patient afflicted with renal cell carcinoma comprising providing to cells of a patient antisense oligonucleotides complementary to polynucleotides encoding one or more kallikrein polypeptides, which are overexpressed in renal cell carcinoma. In an alternative method, expression of genes corresponding to one or more kallikrein polypeptides which are underexpressed in renal cell carcinoma are increased.

The invention relates to a method of inhibiting renal cell carcinoma in a patient at risk for developing renal cell carcinoma comprising inhibiting or increasing expression (or overexpression) of genes encoding kallikrein polypeptides, wherein the kallikrein polypeptides comprise or are selected from the group consisting of kallikrein 5, kallikrein 6, kallikrein 10, and kallikrein 11, that are either overexpressed or underexpressed, in renal cell carcinoma.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The invention relates to newly discovered correlations between expression of certain markers and renal cell carcinoma. The combinations of markers described herein may provide sensitive methods for detecting renal cell carcinoma. The levels of expression of a combination of markers described herein may correlate with the presence of renal cell carcinoma or a pre-malignant condition in a patient. Methods are provided for detecting the presence of renal cell carcinoma in a sample, the absence of renal cell carcinoma in a sample, the stage of a renal cell carcinoma, the grade of a renal cell carcinoma, the benign or malignant nature of a renal cell carcinoma, the metastatic potential of a renal cell carcinoma, assessing the histological type of neoplasm associated with the renal cell carcinoma, the indolence or aggressiveness of the cancer, and other characteristics of renal cell carcinoma that are relevant to prevention, diagnosis, characterization, and therapy of renal cell carcinoma in a patient. Methods are also provided for assessing the efficacy of one or more test agents for inhibiting renal cell carcinoma, assessing the efficacy of a therapy for renal cell carcinoma, monitoring the progression of renal cell carcinoma, selecting an agent or therapy for inhibiting renal cell carcinoma, treating a patient afflicted with renal cell carcinoma, inhibiting renal cell carcinoma in a patient, and assessing the carcinogenic potential of a test compound.

Glossary

The terms “sample”, “biological sample”, and the like, mean a material known or suspected of expressing or containing kallikrien polypeptides associated with renal cell carcinoma, or polynucleotides encoding the polypeptides. The test sample can be used directly as obtained from the source or following a pretreatment to modify the character of the sample. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Nucleic acids and polypeptides may be isolated from the samples and utilized in the methods of the invention. In a preferred embodiment, the sample is a serum sample.

The term “subject” or “patient” refers to a warm-blooded animal such as a mammal, which is suspected of having renal cell carcinoma or a condition, disease, or syndrome associated with renal cell carcinoma. Preferably, “subject” refers to a human.

“Kallikrein polypeptides” or “kallikrein markers” include one or more of kallilkrein 5, kallikrein 6, kallikrein 10, and kallikrein 11. The term includes the native-sequence polypeptides, isoforms, precursors and chimeric polypeptides. The amino acid sequences for native kallikrein polypeptides employed in the present invention include the sequences found in GenBank for each polypeptide as shown in Table 1, and in SEQ ID NO: 1 (kallikrein 5), NO. 4 (kallikrein 6), NO. 8 (kallikrein 10), and NOs. 11 and 12 (kallikrein 11), or a portion thereof. Other useful polypeptides are substantially identical to these sequences (e.g. at least about 45%, preferably 50%, 55%, 60%, 65%, 70%, 75%, 80%, 85%, 90%, 95%, 97%, 98%, or 99% sequence identity), and preferably retain the immunogenic activity of the corresponding native-sequence kallikrein polypeptide.

A “native-sequence polypeptide” comprises a polypeptide having the same amino acid sequence of a polypeptide derived from nature. Such native-sequence polypeptides can be isolated from nature or can be produced by recombinant or synthetic means.

The term “native-sequence polypeptide” specifically encompasses naturally occurring truncated or secreted forms of a polypeptide, polypeptide variants including naturally occurring variant forms (e.g., alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95% amino acid sequence identity to the sequences identified in the GenBank Accession Nos. in Table 1 and shown in SEQ ID NOS 1, 4, 8, 11, and 12. Such variants include, for instance, polypeptides wherein one or more amino acid residues are added to, or deleted from, the N- or C-terminus of the full-length or mature sequences of SEQ ID NOS: 1, 4, 8, 11, and 12, including variants from other species, but excludes a native-sequence polypeptide.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an animo acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a kallikrein polypeptide homolog, for example, the murine kallikrein polypeptide.

Percent identity of two amino acid sequences, or of two nucleic acid sequences identified herein is defined as the percentage of amino acid residues or nucleotides in a candidate sequence that are identical with the amino acid residues in a kallikrein polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

Kallikrien polypeptides include chimeric or fusion proteins. A “chimeric protein” or “fusion protein” comprises all or part (preferably biologically active) of a kallikrein polypeptide operably linked to a heterologous polypeptide (i.e., a polypeptide other than the same kallikrein polypeptide). Within the fusion protein, the term “operably linked” is intended to indicate that the kallikrein polypeptide and the heterologous polypeptide are fused in-frame to each other. The heterologous polypeptide can be fused to the N-terminus or C-terminus of the kallikrein polypeptide. A useful fusion protein is a GST fusion protein in which a kallikrein polypeptide is fused to the C-terminus of GST sequences. Another example of a fusion protein is an immunoglobulin fusion protein in which all or part of a kallikrein polypeptide is fused to sequences derived from a member of the immunoglobulin protein family. Chimeric and fusion proteins can be produced by standard recombinant DNA techniques.

Kallikrein polypeptides may be isolated from a variety of sources, such as from human tissue types or from another source, or prepared by recombinant or synthetic methods, or by any combination of these and similar techniques.

“Kallikrein polynucleotides” or “polynucleotides encoding kallikrein polypeptides” refers to one or more of kallilkrein 5 polynucleotide (KLK5), kallikrein 6 polynucleotide (KLK6), kallikrein 10 polynucleotide (KLK10), and kallikrein 11 polynucleotide (KLK11). The term includes polynucleotides that encode a native-sequence polypeptide, a polypeptide variant including a portion of a kallikrein polypeptide, an isoform, precursor, and chimeric polypeptide.

The polynucleotide sequences encoding native kallikrein polypeptides employed in the present invention include the nucleic acid sequences of the GenBank Accession Nos. identified in Table 1, and in SEQ ID NOs: 2 and 3 (KLK5), NOs. 5, 6, and 7 (KLK6), NOs. 9 and 10 (KLK10), and NOs. 13 and 14 (KLK11), or a fragment thereof.

Polynucleotides encoding kallikrien polypeptides include nucleic acid sequences complementary to these nucleic acids, and polynucleotides that are substantially identical to these sequences (e.g. at least about 45%, preferably 50%, 55%, 60%, 65%, 70%, 75%, 80%, 85%, 90%, 95%, 97%, 98%, or 99% sequence identity).

Kallikrein polynucleotides also include sequences which differ from a nucleic acid sequence of the GenBank Accession Nos. identified in Table 1 and SEQ ID NOS: 2, 3, 5, 6, 7, 9, 10, 13, and 14, due to degeneracy in the genetic code. As one example, DNA sequence polymorphisms within the nucleotide sequence of a kallikrein polypeptide may result in silent mutations that do not affect the amino acid sequence. Variations in one or more nucleotides may exist among individuals within a population due to natural allelic variation. DNA sequence polymorphisms may also occur which lead to changes in the amino acid sequence of a kallikrein polypeptide.

Kallikrein polynucleotides also include nucleic acids that hybridize under stringent conditions, preferably high stringency conditions to a nucleic acid sequence of the GenBank Accession Nos. identified in Table 1 and SEQ ID NOS: 2, 3, 5, 6, 7, 9, 10, 13, and 14. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

Kallikrein polynucleotides also include truncated nucleic acids or nucleic acid fragments and variant forms of the nucleic acids that arise by alternative splicing of an mRNA corresponding to a DNA.

Kallikrien polynucleotides are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method.

One or more kallikrein polypeptides or kallikrein polynucleotides are detected in the present invention. In an aspect, the kallikrein polypeptides comprise kallikrein 6; kallikrein 6 and kallikrein 10; kallikrein 6 and kallikrein 11; kallikrein 6, kallikrein 10 and kallikrein 11; or kallikrein 5, kallikrein 6, kallikrein 10 and kallikrein 11. In another aspect, the kallikrein polynucleotides comprise KLK6; KLK6 and KLK10; KLK6 and KLK11; KLK6, KLK10 and KLK11; or KLK5, KLK6, KLK10 and KLK11

General Methods

A variety of methods can be employed for the diagnostic and prognostic evaluation of renal cell carcinoma involving kallikrein polypeptides, and polynucleotides encoding the polypeptides, and the identification of subjects with a predisposition to such disorders. Such methods may, for example, utilize polynucleotides encoding kallikrein polypeptides, and fragments thereof, and binding agents (e.g. antibodies aptamers) against kallikrein polypeptides, including peptide fragments. In particular, the polynucleotides and antibodies may be used, for example, for (1) the detection of either over- or under-expression of kallikrein polynucleotides, relative to a non-disorder state; and (2) the detection of either an over- or an under-abundance of kallikrein polypeptides, relative to a non-disorder state or the presence of modified (e.g., less than full length) kallikrein polypeptides that correlate with a disorder state, or a progression toward a disorder state.

The invention also contemplates a method for detecting renal cell carcinoma comprising producing a profile of levels of kallikrein polypeptides in cells from a patient, wherein the markers are kallikrein 5, kallikrein 6, kallikrein 10, and/or kallikrein 11, and comparing the profile with a reference to identify a protein profile for the test cells indicative of disease.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring renal cell carcinoma by detecting kallikrein polypeptides, or polynucleotides encoding the polypeptides in biological samples from a subject. These applications require that the amount of polypeptides or polynucleotides quantitated in a sample from a subject being tested be compared to a predetermined standard. The standard may correspond to levels quantitated for another sample or an earlier sample from the subject, or levels quantitated for a control sample. Levels for control samples from healthy subjects or renal cell carcinoma subjects may be established by prospective and/or retrospective statistical studies. Healthy or normal subjects who have no clinically evident disease or abnormalities may be selected for statistical studies. Diagnosis may be made by a finding of statistically different levels of kallikrein polypeptides, or polynucleotides encoding same, compared to a control sample or previous levels quantitated for the same subject. A “significant difference” in levels of markers or polynucleotides in a patient sample compared to a control or standard (e.g. normal levels or levels in other samples from a patient) may represent levels that are higher or lower than the standard error of the detection assay, preferably the levels are at least about 1.5, 2, 3, 4, 5, or 6 times higher or lower, respectively, than the control or standard. The difference in levels of markers or polynucleotides may be a statistically significant difference.

In an embodiment, the levels of kallikrein markers, in particular kallikrein 6 and kallikrein 10, are significantly higher in subjects with renal cell carcinoma, more particularly renal cell carcinoma with tumors of high grade and/or advanced stage. In another embodicment, there is a statsically signifanct difference in kallikrein 6 levels and/or kallikrein 10 levels which are indicative of late stage disease and/or reduced disease survival.

Nucleic Acid Methods/Assays

As noted herein renal cell carcinoma may be detected based on the level of polynucleotides encoding kallikrein polypeptides in a sample. Techniques for detecting nucleic acid molecules such as polymerase chain reaction (PCR) and hybridization assays are well known in the art.

Nucleotide probes for use in the detection of nucleic acid sequences in samples may be constructed using conventional methods known in the art. Suitable probes may be based on nucleic acid sequences encoding at least 5 sequential amino acids from regions of polynucleotides encoding kallikrein polypeptides, preferably they comprise 15 to 40 nucleotides. A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect polynucleotides encoding kallikrein polypeptides preferably in human cells. The nucleotide probes may also be useful in the diagnosis of renal cell carcinoma involving polynucleotides encoding kallikrein polypeptides, in monitoring the progression of such disorder; or monitoring a therapeutic treatment.

Probes may be used in hybridization techniques to detect polynucleotides encoding kallikrein polypeptides. The technique generally involves contacting and incubating nucleic acids (e.g. recombinant DNA molecules, cloned genes) obtained from a sample from a patient or other cellular source with probes under conditions favorable for the specific annealing of the probes to complementary sequences in the nucleic acids. After incubation, the non-annealed nucleic acids are removed, and the presence of nucleic acids that have hybridized to the probe if any are detected.

The detection of polynucleotides encoding kallikrein polypeptides, may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, oligonucleotide primers may be employed in a PCR based assay to amplify a portion of polynucleotides encoding kallikrein polypeptides, derived from a sample, wherein the oligonucleotide primers are specific for (i.e. hybridize to) polynucleotides encoding each of the kallikrein polypeptides. The amplified cDNA is then separated and detected using techniques well known in the art, such as gel electrophoresis.

In order to maximize hybridization under assay conditions, primers and probes employed in the methods of the invention generally have at least about 60%, preferably at least about 75% and more preferably at least about 90% identity to a portion of polynucleotides encoding kallikrein polypeptides. The primers and probes may be at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of expression of polynucleotides encoding kallikrein polypeptides. For example, RNA may be isolated from a cell type or tissue known to express these polynucleotides and tested utilizing the hybridization (e.g. standard Northern analyses) or PCR techniques referred to herein.

The primers and probes may be used in the above-described methods in situ i.e directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample tissue using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to sets of primers specifically designed against a kallikrein polynucleotide sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with suspected renal cell carcinoma and an individual who is not afflicted with renal cell carcinoma. The reaction may be performed on several dilutions of cDNA spanning at least two orders of magnitude. A statistically significant difference in expression in several dilutions of the subject sample as compared to the same dilutions of the non-cancerous sample may be considered positive for the presence of renal cell carcinoma.

Oligonucleotides or longer fragments derived from polynucleotides encoding each kallikrein polypeptide may be used as targets in a microarray. The microarray can be used to simultaneously monitor the expression levels of large numbers of genes. The information from the microarray may be used to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (1995) PCT application WO95/35505; Heller, R. A. et al. (1997) Proc. Natl. Acad. Sci. 94:2150-2155; and Heller, M. J. et al. (1997) U.S. Pat. No. 5,605,662.)

Thus the invention also includes an array comprising polynucleotides encoding kallikrein marker(s). The array can be used to assay expression of kallikrein polynucleotides in the array. The invention allows the quantitation of expression of polynucleotides encoding kallikrein markers.

In an embodiment, the array can be used to monitor the time course of expression of kallikrein polynucleotides in the array. This can occur in various biological contexts such as tumor progression.

The array is also useful for ascertaining differential expression patterns of kallikrein polynucleotides in normal and abnormal cells. This provides a battery of polynucleotides that could serve as molecular targets for diagnosis or therapeutic intervention.

Protein Methods

Binding agents specific for kallikrein polypeptides may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of an renal cell carcinoma in a subject may be determined by (a) contacting a sample from the subject with binding agents for kallikrein polypeptides; (b) detecting in the sample levels of polypeptides that bind to the binding agents; and (c) comparing the levels of polypeptides with a predetermined standard or cut-off value.

“Binding agent” refers to a substance such as a polypeptide or antibody that specifically binds to a kallikrein polypeptide. A substance “specifically binds” to a polypeptide if it reacts at a detectable level with the kallikrein polypeptide, and does not react detectably with peptides containing unrelated sequences or sequences of different polypeptides. Binding properties may be assessed using an ELISA, which may be readily performed by those skilled in the art (see for example, Newton et al, Develop. Dynamics 197: 1-13, 1993).

A binding agent may be a ribosome, with or without a peptide component, an aptamer, an RNA molecule, or a polypeptide. A binding agent may be a polypeptide that comprises a kallikrein polypeptide sequence, a peptide variant thereof, or a non-peptide mimetic of such a sequence. By way of example a kallikrein polypeptide sequence may be a peptide portion of a kallikrein polypeptide that is capable of modulating a function mediated by the kallikrein polypeptide.

An aptamer includes a DNA or RNA molecule that binds to polynucleotides and polypeptides. An aptamer that binds to a polypeptide (or binding domain) of a kallikrein polypeptide or a polynucleotide encoding a kallikrein polypeptide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

In certain other preferred embodiments, the binding agent is an antibody.

In an aspect the present invention provides a diagnostic method for monitoring or diagnosing renal cell carcinoma in a subject by quantitating kallikrein polypeptides, in a biological sample from the subject comprising reacting the sample with antibodies specific for kallikrein polypeptides, which are directly or indirectly labelled with detectable substances, and detecting the detectable substances.

In an aspect of the invention, a method for detecting renal cell carcinoma is provided comprising:
(a) obtaining a sample suspected of containing kallikrein polypeptides associated with renal cell carcinoma, wherein the kallikrein polypeptides comprise or are selected from the group consisting of kallikrein 5, kallikrein 6, kallikrein 10 and kallikrein 11;
(b) contacting the sample with antibodies that specifically bind kallikrein polypeptides, under conditions effective to bind the antibodies and form complexes;
(c) measuring the amount of kallikrein polypeptides, present in the sample by quantitating the amount of the complexes; and
(d) comparing the amount of kallikrein polypeptides, present in the samples with the amount of polypeptides in a control, wherein a change or significant difference in the amount of polypeptides in the sample compared with the amount in the control is indicative of renal cell carcinoma.

In an embodiment, the invention contemplates a method for monitoring the progression of renal cell carcinoma in an individual, comprising:
(a) contacting antibodies which bind to kallikrein polypeptides, with a sample from the individual so as to form binary complexes comprising each of the antibodies and polypeptides in the sample, wherein the kallikrein polypeptides comprise or are selected from the group consisting of kallikrein 5, kallikrein 6, kallikrein 10 and kallikrein 11;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of the stage and/or progression of the renal cell carcinoma in said individual.

The amount of complexes may also be compared to a value representative of the amount of the complexes from an individual not at risk of, or afflicted with, renal cell carcinoma at different stages.

Thus, antibodies specifically reactive with a kallikrein polypeptide, or derivatives, such as enzyme conjugates or labeled derivatives, may be used to detect a kallikrein polypeptide, in various samples (e.g. biological materials). They may be used as diagnostic or prognostic reagents and they may be used to detect abnormalities in the levels of expression of kallikrein polypeptides, or abnormalities in the structure, and/or temporal, tissue, cellular, or subcellular location of kallikrein polypeptides. Antibodies may also be used to screen potentially therapeutic compounds in vitro to determine their effects on renal cell carcinoma involving kallikrein-polypeptides, and other conditions. In vitro immunoassays may also be used to assess or monitor the efficacy of particular therapies.

Antibodies may be used in any known immunoassays that rely on the binding interaction between antigenic determinants of kallikrein polypeptides, and the antibodies. Examples of such assays are radioimmunoassays, enzyme immunoassays (e.g. ELISA), immunofluorescence, immunoprecipitation, latex agglutination, hemagglutination, and histochemical tests. These terms are well understood by those skilled in the art. A person skilled in the art will know, or can readily discern, other immunoassay formats without undue experimentation.

In particular, the antibodies may be used in immunohistochemical analyses, for example, at the cellular and sub-subcellular level, to detect kallikrein polypeptides, to localize them to particular renal cell carcinoma tumor cells and tissues, and to specific subcellular locations, and to quantitate the level of expression.

Antibodies for use in the present invention include monoclonal or polyclonal antibodies, immunologically active fragments (e.g. a Fab or (Fab)₂ fragments), antibody heavy chains, humanized antibodies, antibody light chains, genetically engineered single chain F, molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant kallikrein polypeptides may be utilized to prepare antibodies. See, for example, Kohler et al. (1975) Nature 256:495-497; Kozbor et al. (1985) J. Immunol Methods 81:3142; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. The antibodies specific for kallikrein polypeptides used in the methods of the invention may also be obtained from scientific or commercial sources.

In an embodiment of the invention, antibodies are reactive against kallikrein polypeptides if they bind with a Kₐ of greater than or equal to 10⁻⁷ M.

Antibodies that bind to kallikrein polypeptides may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against a kallikrein polypeptide. The second antibody may be labeled with a detectable substance to detect the primary antigen-antibody reaction. By way of example, if the antibody having specificity against a kallikrein polypeptide is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect kallikrein polypeptides. Generally, antibodies may be labeled with detectable substances and kallikrein polypeptides, may be localised in tissues and cells based upon the presence of the detectable substance.

In the context of the methods of the invention, the sample, binding agents (e.g. antibodies) for kallikrein polypeptides may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for a kallikrein polypeptide may be immobilized using sheep anti-mouse IgG Fc fragment specific antibody coated on the carrier or support.

Where radioactive labels are used as a detectable substance, kallikrein polypeptides may be localized by radioautography. The results of radioautography may be quantitated by determining the density of particles in the radioautographs by various optical methods, or by counting the grains.

Time-resolved fluorometry may be used to detect a signal. For example, the method described in Christopoulos T K and Diamandis E P Anal Chem 1992:64:342-346 may be used with a conventional time-resolved fluorometer.

Therefore, in accordance with an embodiment of the invention, a method is provided wherein antibodies specific for each kallikrein polypeptide, are labelled with enzymes, substrates for the enzymes are added wherein the substrates are selected so that the substrates, or a reaction product of the enzymes and substrates, form fluorescent complexes with lanthanide metals. Lanthanide metals are added and the kallikrein polypeptides are quantitated in the sample by measuring fluorescence of the fluorescent complexes. Antibodies specific for the kallikrein polypeptides may be directly or indirectly labelled with enzymes. Enzymes are selected based on the ability of a substrate of the enzyme, or a reaction product of the enzyme and substrate, to complex with lanthanide metals such as europium and terbium. Examples of suitable enzymes include alkaline phosphatase and β-galactosidase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

Antibodies specific for kallikrein polypeptides may also be indirectly labelled with enzymes. For example, an antibody may be conjugated to one partner of a ligand binding pair, and the enzyme may be coupled to the other partner of the ligand binding pair. Representative examples include avidin-biotin, and riboflavin-riboflavin binding protein. In another embodiment, antibodies specific for the anti-kallikrein antibodies are labeled with an enzyme.

In accordance with an embodiment, the present invention provides means for determining kallikrein polypeptides in a sample, in particular a serum sample, by measuring kallikrein polypeptides by immunoassay. It will be evident to a skilled artisan that a variety of immunoassay methods can be used to measure kallikrein polypeptides in serum. In general, an immunoassay method may be competitive or noncompetitive. Competitive methods typically employ immobilized or immobilizable antibodies to the kallikrein polypeptides and a labeled form of each of the kallikrein polypeptides. Kallikrein polypeptides and labeled kallikrein polypeptides compete for binding to anti-kallikrein antibodies. After separation of the resulting labeled kallikrein polypeptides that have become bound to anti-kallikrein polypeptides (bound fraction) from that which has remained unbound (unbound fraction), the amount of the label in either bound or unbound fraction is measured and may be correlated with the amount of kallikrein polypeptides, in the test sample in any conventional manner, e.g., by comparison to a standard curve.

In an aspect, a non-competitive method is used for the determination of kallikrein polypeptides with the most common method being the “sandwich” method. In this assay, two types of antibodies specific for kallikrein polypeptides are employed. One type of antibody is directly or indirectly labeled (sometimes referred to as the “detection antibody”) and the other is immobilized or immobilizable (sometimes referred to as the “capture antibody”). The capture and detection antibodies can be contacted simultaneously or sequentially with a test sample. Sequential methods can be accomplished by incubating capture antibodies with the sample, and adding the detection antibodies at a predetermined time thereafter (sometimes referred to as the “forward” method); or the detection antibodies can be incubated with the sample first and then the capture antibodies added (sometimes referred to as the “reverse” method). After the necessary incubation(s) have occurred, to complete the assay, the capture antibodies are separated from the liquid test mixture, and labels are measured in at least a portion of the separated capture antibody phase or the remainder of the liquid test mixture. Generally the labels are measured in the capture antibody phase since it comprises kallikrein polypeptides bound by (“sandwiched” between) the capture and detection antibodies. In an embodiment, the label may be measured without separating the capture antibodies and liquid test mixture.

In a typical two-site immunometric assay for kallikrein polypeptides, one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The labels used with the detection antibodies can be selected from any of those known conventionally in the art. The labels may be an enzyme or a chemiluminescent moiety, but it can also be a radioactive isotope, a fluorophor, a detectable ligand (e.g., detectable by a secondary binding by a labeled binding partner for the ligand), and the like. Preferably antibodies are labelled with enzymes which are detected by adding substrates that are selected so that a reaction product of the enzymes and substrates forms fluorescent complexes. Capture antibodies may be selected so that they provide a means for being separated from the remainder of the test mixture. Accordingly, the capture antibodies can be introduced to the assay in an already immobilized or insoluble form, or can be in an immobilizable form, that is, a form which enables immobilization to be accomplished subsequent to introduction of the capture antibodies to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, a capture antibody may be immobilized using a species specific antibody for the capture antibody that is bound to the solid phase.

A particular sandwich immunoassay method of the invention employs two types of antibodies, first antibodies are reactive against kallikrein polypeptides and second antibodies having specificity against antibodies reactive against kallikrein polypeptides labelled with enzymatic labels, and fluorogenic substrates for the enzymes. An enzyme may be alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb3+ fluorescence in a time-resolved mode. Fluorescence intensity is measured using a time-resolved fluorometer as described herein.

The above-described immunoassay methods and formats are intended to be exemplary and are not limiting.

Computer Systems

Computer readable media comprising kallikrein markers is also provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on kallikrein markers.

A variety of data processor programs and formats can be used to store information on kallikrein markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has renal cell carcinoma or a pre-disposition to renal cell carcinoma, comprising determining the presence or absence of kallikrein markers or polynucleotides encoding same, and based on the presence or absence of the kallikrein markers or polynucleotides encoding same, determining whether the patient has renal cell carcinoma or a pre-disposition to renal cell carcinoma, and optionally recommending treatment for the renal cell carcinoma or pre-renal cell carcinoma condition.

The invention also provides in an electronic system and/or in a network, a method for determining whether a subject has renal cell carcinoma or a pre-disposition to renal cell carcinoma associated with kallikrein markers or polynucleotides encoding same, comprising determining the presence or absence of kallikrein markers or polynucleotides encoding same, and based on the presence or absence of the kallikrein markers or polynucleotides encoding same, determining whether the subject has renal cell carcinoma or a pre-disposition to renal cell carcinoma, and optionally recommending treatment for the renal cell carcinoma or pre-renal cell carcinoma condition.

The invention further provides in a network, a method for determining whether a subject has renal cell carcinoma or a pre-disposition to renal cell carcinoma associated with kallikrein markers or polynucleotides encoding same, comprising: (a) receiving phenotypic information on the subject and information on kallikrein markers or polynucleotides encoding same, associated with samples from the subject; (b) acquiring information from the network corresponding to the kallikrein markers or polynucleotides encoding same; and (c) based on the phenotypic information and information on the kallikrein markers or polynucleotides encoding same, determining whether the subject has renal cell carcinoma or a pre-disposition to renal cell carcinoma; and (d) optionally recommending treatment for the renal cell carcinoma or pre-renal cell carcinoma condition.

The invention still further provides a system for identifying selected records that identify a renal cell carcinoma cell. A system of the invention generally comprises a digital computer; a database server coupled to the computer, a database coupled to the database server having data stored therein, the data comprising records of data comprising kallikrein markers, or polynucleotides encoding same, and a code mechanism for applying queries based upon a desired selection criteria to the data file in the database to produce reports of records which match the desired selection criteria.

In an aspect of the invention a method is provided for detecting a renal cell carcinoma cell using a computer having a processor, memory, display, and input/output devices, the method comprising the steps of:
(a) creating records of kallikrein markers or polynucleotides encoding same, isolated from a sample suspected of containing an renal cell carcinoma cell;
(b) providing a database comprising records of data comprising kallikrein markers, wherein the markers are kallikrein 5, kallikrein 6, kallikrein 10, and kallikrein 11, and/or comprising polynucleotides encoding same; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from a cell that is a renal cell carcinoma cell.

The invention contemplates a business method for determining whether a subject has renal cell carcinoma or a pre-disposition to renal cell carcinoma associated with kallikrein markers or polynucleotides encoding same comprising: (a) receiving phenotypic information on the subject and information on kallikrein markers or polynucleotides encoding same, associated with samples from the subject; (b) acquiring information from a network corresponding to the kallikrein markers or polynucleotides encoding same; and (c) based on the phenotypic information, information on the kallikrein markers or polynucleotides encoding same, and acquired information, determining whether the subject has renal cell carcinoma or a pre-disposition to renal cell carcinoma; and (d) optionally recommending treatment for the renal cell carcinoma or pre-renal cell carcinoma condition.

Imaging Methods

Antibodies specific for kallikrein polypeptides may also be used in imaging methodologies in the management of renal cell carcinoma. The invention provides a method for imaging tumors associated with kallikrein polypeptides.

In an embodiment the method is an in vivo method and a subject or patient is administered imaging agents that carry imaging labels and are capable of targeting or binding to kallikrein polypeptides. In the method each imaging agent is labeled so that it can be distinguished during the imaging. The imaging agents are allowed to incubate in vivo and bind to the kallikrein polypeptides associated with renal cell carcinoma tumors. The presence of label is localized to the renal cell carcinoma, and the localized label is detected using imaging devices known to those skilled in the art.

The imaging agents may be antibodies or chemical entities that recognize kallikrein polypeptides. In an aspect of the invention an imaging agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for kallikrein polypeptides used in the methods of the invention may be obtained from scientific or commercial sources, or isolated native or recombinant kallikrein polypeptides may be utilized to prepare antibodies etc as described herein.

An imaging agent may be a peptide that mimics the epitope for an antibody specific for a kallikrein polypeptide and binds to a kallikrein polypeptide. The peptide may be produced on a commercial synthesizer using conventional solid phase chemistry. By way of example, a peptide may be prepared that includes tyrosine, lysine, or phenylalanine to which N₂S₂ chelate is complexed (See U.S. Pat. No. 4,897,255). The anti-kallikrein peptide conjugate is then combined with a radiolabel (e.g. sodium ^{99m}Tc pertechnetate or sodium ¹⁸⁸Re perrhenate) and it may be used to locate a tumor producing the kallikrein polypeptides.

Imaging agents carry labels to image the kallikrein polypeptides. Agents may be labelled for use in radionuclide imaging. In particular, agents may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, 72Ga, ¹⁹⁸Au, ³H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ^{191m-191}Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁵Y, ⁹⁰Y, ⁶²Zn and ⁶⁵Zn. Preferably the radioisotope is ¹³¹I, ¹²¹I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl ⁷⁷Br, or ¹³F, and it is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for murine monoclonal antibodies are described by Goding, J. W. (supra, pp 124-126) and the references cited therein. Other procedures for iodinating biological agents, such as antibodies, binding portions thereof, probes, or ligands, are described in the scientific literature (see Hunter and Greenwood, Nature 144:945 (1962), David et al., Biochemistry 13:1014-1021 (1974), and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). ^{99m} Tc-labeling procedures are described by Rhodes, B. et al. in Burchiel, S. et al., (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat. No. 4,311,688. Procedures suitable for ¹¹¹In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

An imaging agent may also be labeled with a paramagnetic isotope for purposes of an in vivo method of the invention. Examples of elements that are useful in magnetic resonance imaging include gadolinium, terbium, tin, iron, or isotopes thereof (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest Radiol. 19, 408-415 for discussions on in vivo nuclear magnetic resonance imaging.)

In the case of radiolabeled agents, the agents may be administered to the patient, localized to the tumor having one or more kallikrein polypeptides with which the agents bind, and detected or “imaged” in vivo using known techniques such as radionuclear scanning using, for example, a gamma camera or emission tomography. [See for example, A. R P Bradwell et al., “Developments in Antibody Imaging”, Monoclonal Antibodies for Cancer Detection and Therapy, R. W. Baldwin et al., (eds.), pp. 65-85 (Academic Press 1985)]. A positron emission transaxial tomography scanner, such as designated Pet VI located at Brookhaven National Laboratory, can also be used where the radiolabel emits positrons (e.g., ¹¹C, ¹⁸F, ¹⁵O, and ¹³N).

Whole body imaging techniques using radioisotope labeled agents can be used for locating both primary tumors and tumors which have metastasized. Antibodies specific for kallikrein polypeptides or fragments thereof having the same epitope specificity, are bound to a suitable radioisotope, or a combination thereof, and administered parenterally. For renal cell carcinoma, administration preferably is intravenous. The bio-distribution of the labels can be monitored by scintigraphy, and accumulations of the labels can be related to the presence of renal cell carcinoma cells. Whole body imaging techniques are described in U.S. Pat. Nos. 4,036,945 and 4,311,688. Other examples of agents useful for diagnosis and therapeutic use that can be coupled to antibodies and antibody fragments include metallothionein and fragments (see, U.S. Pat. No. 4,732,864). These agents are useful in diagnosis, staging and visualization of cancer, in particular renal cell carcinoma, so that surgical and/or radiation treatment protocols can be used more efficiently.

Screening Methods

The invention also contemplates methods for evaluating test agents or compounds for their ability to inhibit renal cell carcinoma or potentially contribute to renal cell carcinoma. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)2, and Fab expression library fragments, and epitope-binding fragments thereof)], nucleic acids (e.g. antisense interference RNA) and small organic or inorganic molecules. The agents or compounds may be endogenous physiological compounds or natural or synthetic compounds.

The invention also provides a method for assessing the potential efficacy of a test agent for inhibiting renal cell carcinoma in a patient, the method comprising comparing:
(a) levels of kallikrein markers or polynucleotides encoding same in a first sample obtained from a patient and exposed to the test agent, wherein the markers comprise or are selected from the group consisting of kallikrein 5, kallikrein 6, kallikrein 10, and kallikrein 11, and
(b) levels of the kallikrein markers or polynucleotides encoding same in a second sample obtained from the patient, wherein the sample is not exposed to the test agent, wherein a significant difference in the levels of expression of kallikrein markers or polynucleotides encoding same in the first sample, relative to the second sample, is an indication that the test agent is potentially efficacious for inhibiting renal cell carcinoma in the patient.

The first and second samples may be portions of a single sample obtained from a patient or portions of pooled samples obtained from a patient.

In an aspect, the invention provides a method of selecting an agent for inhibiting renal cell carcinoma in a patient comprising:
(a) obtaining a sample comprising cancer cells from the patient;
(b) separately maintaining aliquots of the sample in the presence of a plurality of test agents;
(c) comparing kallikrein markers and/or polynucleotides encoding same, in each of the aliquots, wherein the markers comprise or are selected from the group consisting of kallikrein 5, kallilkrein 6, kallikrein 10, and kallikrein 11; and
(d) selecting one of the test agents which alters the levels of the kallikrein markers and/or polynucleotides encoding same in the aliquot containing that test agent, relative to other test agents.

Still another aspect of the present invention provides a method of conducting a drug discovery business comprising:
(a) providing one or more methods or assay systems for identifying agents that inhibit renal cell carcinoma in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the renal cell carcinoma carcinogenic potential of a test compound comprising:
(a) maintaining separate aliquots of renal cell carcinoma cells in the presence and absence of the test compound; and
(b) comparing kallikrein markers and/or polynucleotides encoding same in each of the aliquots, wherein the markers comprise or are selected from the group consisting of kallikrein 5, kallikrein 6, kallikrein 10, and kallikrein 11.

A significant difference between the levels of the markers in the aliquot maintained in the presence of (or exposed to) the test compound relative to the aliquot maintained in the absence of the test compound, indicates that the test compound possesses renal cell carcinoma carcinogenic potential.

Kits

The methods described herein may be performed by utilizing pre-packaged diagnostic kits comprising at least kallikrein polynucleotides or binding agents (e.g. antibodies) described herein, which may be conveniently used, e.g., in clinical settings, to screen and diagnose patients, and to screen and identify those individuals afflicted with or exhibiting a predisposition to renal cell carcinoma.

Thus, the invention also contemplates kits for carrying out the methods of the invention. Such kits typically comprise two or more components required for performing a diagnostic assay. Components include but are not limited to compounds, reagents, containers, and/or equipment.

In an embodiment, a container with a kit comprises binding agents as described herein. By way of example, the kit may contain antibodies specific for kallikrein polypeptides, antibodies against the antibodies labelled with enzymes, and substrates for the enzymes. The kit may also contain microtiter plate wells, standards, assay diluent, wash buffer, adhesive plate covers, and/or instructions for carrying out a method of the invention using the kit.

In an aspect of the invention, the kit includes antibodies or antibody fragments which bind specifically to epitopes of kallikrein polypeptides, and means for detecting binding of the antibodies to epitopes associated with tumor cells, either as concentrates (including lyophilized compositions), which nay be further diluted prior to use or at the concentration of use, where the vials may include one or more dosages. Where the hits are intended for in vivo use, single dosages may be provided in sterilized containers, having the desired amount and concentration of agents. Containers that provide a formulation for direct use, usually do not require other reagents, as for example, where the kit contains radiolabelled antibody preparations for in vivo imaging.

A kit may be designed to detect the level of polynucleotides encoding kallikrein polypeptides in a sample. Such kits generally comprise oligonucleotide probes or primers, as described herein, that hybridize to polynucleotides encoding kallikrein polypeptides. Such oligonucleotides may be used, for example, within a PCR or hybridization procedure. Additional components that may be present within the kits include second oligonucleotides and/or diagnostic reagents to facilitate detection of polynucleotides encoding kallikrein polypeptides.

The reagents suitable for applying the screening methods of the invention to evaluate compounds may be packaged into convenient kits described herein providing the necessary materials packaged into suitable containers.

Applications

Kallikrein polypeptides are targets for renal cell carcinoma immunotherapy. Such immunotherapeutic methods include the use of antibody therapy, in vivo vaccines, and ex vivo immunotherapy approaches.

In one aspect, the invention provides antibodies specific for kallikrein polypeptides that may be used systemically to treat renal cell carcinoma. Preferably antibodies are used that target the tumor cells but not the surrounding non-tumor cells and tissue. Thus, the invention provides a method of treating a patient susceptible to, or having a cancer that expresses kallikrein polypeptides comprising administering to the patient an effective amount of antibodies that bind specifically to kallikrein polypeptides. In another aspect, the invention provides a method of inhibiting the growth of tumor cells expressing kallikrein polypeptides, comprising administering to a patient antibodies which bind specifically to kallikrein polypeptides in amounts effective to inhibit growth of the tumor cells. Antibodies specific for kallikrein polypeptides may also be used in a method for selectively inhibiting the growth of, or killing a cell expressing kallikrein polypeptides comprising reacting antibody immunoconjugates or immunotoxins with the cell in an amount sufficient to inhibit the growth of, or kill the cell.

By way of example, unconjugated antibodies specific for kallikrein polypeptides may be introduced into a patient such that the antibodies bind to cancer cells expressing kallikrein polypeptides and mediate growth inhibition of such cells (including the destruction thereof), and the tumor, by mechanisms which may include complement-mediated cytolysis, antibody-dependent cellular cytotoxicity, altering the physiologic function of kallikrein polypeptides and/or the inhibition of ligand binding or signal transduction pathways. In addition to unconjugated antibodies, antibodies specific for kallikrein polypeptides, conjugated to therapeutic agents (e.g. immunoconjugates) may also be used therapeutically to deliver the agents directly to tumor cells expressing kallikrein polypeptides and thereby destroy the tumor. Examples of such agents include abrin, ricin A, Pseudomonas exotoxin, or diphtheria toxin, proteins such as tumor necrosis factor, alpha-interferon, beta-interferon, nerve growth factor, platelet derived growth factor, tissue plasminogen activator, and biological response modifiers such as lymphokines, interleukin-1, interleukin-2, interleukin-6, granulocyte macrophage colony stimulating factor, granulocyte colony stimulating factor, or other growth factors.

Cancer immunotherapy using antibodies specific for kallikrein polypeptides may utilize the various approaches that have been successfully employed for cancers, including but not limited to colon cancer (Arlen et al., 1998, Crit Rev Immunol 18: 133-138), multiple myeloma (Ozaki et al., 1997, Blood 90: 3179-3186; Tsunenati et al., 1997, Blood 90: 2437-2444), gastric cancer (Kasprzyk et al., 1992, Cancer Res 52: 2771-2776), B-cell lymphoma (Funakoshi et al., 1996, J Immunther Emphasis Tumor Immunol 19: 93-101), leukemia (Zhong et al., 1996, Leuk Res 20: 581-589), colorectal cancer (Moun et al., 1994, Cancer Res 54: 6160-6166); Velders et al., 1995, Cancer Res 55: 4398-4403), and breast cancer (Shepard et al., 1991, J Clin Immunol 11: 117-127).

In the practice of a method of the invention, antibodies specific for kallikrein polypeptides capable of inhibiting the growth of cancer cells expressing kallikrein polypeptides are administered in a therapeutically effective amount to cancer patients whose tumors express or overexpress kallikrein polypeptides. The invention may provide a specific, effective and long-needed treatment for renal cell carcinoma. The antibody therapy methods of the invention may be combined with other therapies including chemotherapy and radiation.

Patients may be evaluated for the presence and levels of kallikrein polypeptides expression and overexpression in tumors, preferably using immunohistochemical assessments of tumor tissue, quantitative imaging as described herein, or other techniques capable of reliably indicating the presence and degree of expression of kallikrein polypeptides. Immunohistochemical analysis of tumor biopsies or surgical specimens may be employed for this purpose.

Antibodies specific for kallikrein polypeptides useful in treating cancer include those that are capable of initiating a potent immune response against the tumor and those that are capable of direct cytotoxicity. In this regard, the antibodies may elicit tumor cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. In addition, antibodies specific for kallikrein polypeptides that exert a direct biological effect on tumor growth are useful in the practice of the invention. Such antibodies may not require the complete immunoglobulin to exert the effect. Potential mechanisms by which such directly cytotoxic antibodies may act include inhibition of cell growth, modulation of cellular differentiation, modulation of tumor angiogenesis factor profiles, and the induction of apoptosis. The mechanism by which a particular antibody exerts an anti-tumor effect may be evaluated using any number of in vitro assays designed to determine ADCC, antibody-dependent macrophage-mediated cytotoxicity (ADMMC), complement-mediated cell lysis, and others known in the art.

The anti-tumor activity of a combination of antibodies specific for kallikrein polypeptides may be evaluated in vivo using a suitable animal model. Xenogenic cancer models, wherein human cancer explants or passaged xenograft tissues are introduced into immune compromised animals, such as nude or SCID mice, may be employed.

The methods of the invention contemplate the administration of combinations, or “cocktails” of different individual antibodies recognizing epitopes of kallikrein polypeptides. Such cocktails may have certain advantages inasmuch as they contain antibodies that bind to different epitopes and/or exploit different effector mechanisms or combine directly cytotoxic antibodies with antibodies that rely on immune effector functionality. Such antibodies in combination may exhibit synergistic therapeutic effects. In addition, the administration of the antibodies may be combined with other therapeutic agents, including but not limited to chemotherapeutic agents, androgen-blockers, and immune modulators (e.g., IL2, GM-CSF). The antibodies may be administered in their “naked” or unconjugated form, or may have therapeutic agents conjugated to them.

The antibodies specific for kallikrein polypeptides used in the practice of the method of the invention may be formulated into pharmaceutical compositions comprising a carrier suitable for the desired delivery method. Suitable carriers include any material which when combined with the antibodies retains the anti-tumor function of the antibodies and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

Antibody formulations may be administered via any route capable of delivering the antibodies to the tumor site. Routes of administration include, but are not limited to, intravenous, intraperitoneal, intramuscular, intratumor, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of cancer and the severity, grade, or stage of the cancer, the binding affinity and half life of the antibodies used, the degree of expression of kallikrein polypeptides in the patient, the extent of circulating kallikrein polypeptide antigens the desired steady-state antibody concentration level, frequency of treatment, and the influence of any chemotherapeutic agents used in combination with a treatment method of the invention.

Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of antibodies necessary to be therapeutically effective in a particular context. Repeated administrations may be required to achieve tumor inhibition or regression. Direct administration of antibodies specific for kallikrein polypeptides is also possible and may have advantages in certain situations.

Patients may be evaluated for kallikrein polypeptides, preferably in serum, in order to assist in the determination of the most effective dosing regimen and related factors. The assay methods described herein, or similar assays, may be used for quantitating circulating kallikrein polypeptide levels in patients prior to treatment. Such assays may also be used for monitoring throughout therapy, and may be useful to gauge therapeutic success in combination with evaluating other parameters, such as serum kallikrein polypeptides.

The invention further provides vaccines formulated to contain kallikrein polypeptides or fragments thereof. The use in anti-cancer therapy of tumor antigens in a vaccine for generating humoral and cell-mediated immunity is well known and, for example, has been employed in prostate cancer using human PSMA and rodent PAP immunogens (Hodge et al., 1995, Int. J. Cancer 63: 231-237; Fong et al., 1997, J. Immunol. 159: 3113-3117). These methods can be practiced by employing kallikrein polypeptides, or fragments thereof, or polynucleotides and recombinant vectors capable of expressing and appropriately presenting the kallikrein immunogens.

By way of example, viral gene delivery systems may be used to deliver polynucleotides encoding kallikrein polypeptides. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding kallikrein polypeptides, or fragments thereof introduced into the patient (e.g., intramuscularly) to induce an anti-tumor response.

Various ex vivo strategies may also be employed. One approach involves the use of cells to present kallikrein antigens to a patient's immune system. For example, autologous dendritic cells which express MHC class I and II, may be pulsed with kallikrein polypeptides, or peptides thereof that are capable of binding to MHC molecules, to thereby stimulate renal cell carcinoma patients' immune systems (See, for example, Tjoa et al., 1996, Prostate 28: 65-69; Murphy et al., 1996, Prostate 29: 371-380).

Anti-idiotypic antibodies specific for kallikrein polypeptides can also be used in anti-cancer therapy as a vaccine for inducing an immune response to cells expressing the polypeptides. The generation of anti-idiotypic antibodies is well known in the art and can readily be adapted to generate anti-idiotypic antibodies that mimic an epitope on a kallikrein polypeptide (see, for example, Wagner et al., 1997, Hybridoma 16: 33-40; Foon et al., 1995, J Clin Invest 96: 334-342; Herlyn et al., 1996, Cancer Immunol Immunother 43: 65-76). Such antibodies can be used in anti-idiotypic therapy as presently practiced with other anti-idiotypic antibodies directed against tumor antigens.

Genetic immunization methods may be utilized to generate prophylactic or therapeutic humoral and cellular immune responses directed against cancer cells expressing kallikrein polypeptides. Constructs comprising DNA encoding kallikrein polypeptides/immunogens and appropriate regulatory sequences may be injected directly into muscle or skin of an individual, such that the cells of the muscle or skin take-up the construct and express the encoded kallikrein polypeptides/immunogens. The polypeptides/immunogens may be expressed as cell surface proteins or be secreted. Expression of the polypeptides/immunogens results in the generation of prophylactic or therapeutic humoral and cellular immunity against the cancer. Various prophylactic and therapeutic genetic immunization techniques known in the art may be used.

The invention further provides methods for inhibiting cellular activity (e.g., cell proliferation, activation, or propagation) of a cell expressing kallikrein polypeptides. This method comprises reacting immunoconjugates of the invention (e.g., a heterogeneous or homogenous mixture) with the cell so that the kallikrein polypeptides form a complex with the immunoconjugates. A subject with a neoplastic or preneoplastic condition can be treated when the inhibition of cellular activity results in cell death.

In another aspect, the invention provides methods for selectively inhibiting a cell expressing kallikrein polypeptides by reacting a combination of the immunoconjugates of the invention with the cell in an amount sufficient to inhibit the cell. Amounts include those that are sufficient to kill the cell or sufficient to inhibit cell growth or proliferation.

Vectors derived from retroviruses, adenovirus, herpes or vaccinia viruses, or from various bacterial plasmids, may be used to deliver polynucleotides encoding kallikrein polypeptides to a targeted organ, tissue, or cell population. Methods well known to those skilled in the art may be used to construct recombinant vectors that will express antisense nucleic acid molecules for kallikrein polypeptides. (See, for example, the techniques described in Sambrook et al (supra) and Ausubel et al (supra)).

Genes encoding kallikrein polypeptides can be turned off by transfecting a cell or tissue with vectors that express high levels of a desired kallikrein polypeptide-encoding fragments. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of genes encoding kallikrein polypeptides, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding kallikrein polypeptides.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

Kallikrein polypeptides and polynucleotides encoding the polypeptides, and fragments thereof, antibodies, and/or agents identified using a method of the invention, or combinations thereof, may be used in the treatment of renal cell carcinoma or diseases, conditions or syndromes associated with renal cell carcinoma, in a subject. The kallikrein polypeptides, polynucleotides, and agents may be formulated into compositions for administration to subjects suffering from renal cell carcinoma. Therefore, the present invention also relates to a composition comprising kallikrein polypeptides, or polynucleotides encoding the polypeptides, or a fragment thereof, or an agent identified using a method of the invention, and a pharmaceutically acceptable carrier, excipient or diluent. A method for treating or preventing renal cell carcinoma in a subject is also provided comprising administering to a patient in need thereof, kallikrein polypeptides, or polynucleotides encoding the polypeptides, an agent identified in accordance with a method of the invention, or a composition of the invention.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment (e.g. chemotherapy or radiotherapy). The compositions of the invention may be administered concurrently, separately, or sequentially with other therapeutic agents or therapies.

The following non-limiting examples are illustrative of the present invention:

### EXAMPLE 1

Human kallikreins 5, 6, 10, and 11 (hK5,/6/10/11) are expressed by many normal tissues and it has been suggested that they may represent candidate cancer diagnostic/prognostic markers. Renal cell carcinoma (RCC) is characterized by unpredictable outcome, despite the use of conventional prognostic factors. The immunohistochemical expression (IE) and the predictive value of the above kallikreins was evaluated in RCC. Included in the study were 95 patients who underwent radical nephrectomy for RCC. The median follow-up was 60 (1-180) months. Fifty seven of the 95 RCC cases were immunostained for hK5, 70/95 for hK6, 70/95 for hK10, and 69/95 for hK11, respectively. The immunohistochemical method of streptavidin-biotin-peroxidase using anti-hK5,/6/10/11 monoclonal and polyclonal antibodies was performed. The IE of all kallikreins was correlated with tumor size, histological type, histological malignancy, mitotic index, and pathological stage. The Fuhrman 4-scale grading system was used for the determination of the histological malignancy. For statistical analysis, four grades were collapsed into two and RCC cases were categorized as low and high malignant (LM and HM) respectively. Statistical analysis included Pearson Chi-square and Kruskal Wallis tests. Survival data were analyzed using Kaplan-Meier and Cox regression analysis. A Pvalue<0.05 was considered significant. In the normal (adjacent to tumor) renal parenchyma, the epithelium of the urinary tubuli showed a cytoplasmic IE of all kallikreins. In RCC, their IE was decreased: 33/57 cases (58%) were positive for hK5, 27/70 (39%) for hK6, 46/70 (66%) for hK10 and 32/69 (46%) for hK11, respectively. A statistically significant positive correlation was observed among the IE of all kallikreins. HM-RCC expressed all kallikreins in a higher percentage than LM-RCC, but a statistically significant difference was observed only in the case of hK6 and hB10 (55% vs. 27%, p=0.016 and 79% vs. 56%, p=0.044 respectively). Hk6 and hK11 showed a positive correlation with pathological stage: hK6 with both Robson and TNM 1997 staging systems (p=0.010 and p=0.017 respectively), and hK11 only with the Robson staging system (p=0.045). In both the Kaplan-Meier and the univariate Cox regression analysis, hK6 IE showed a negative correlation with disease survival (p=0.05 and p=0.038 respectively). In univariate analysis, nuclear grade, Robson and TNM staging systems correlated with disease survival as well. However, in the multivariate analysis, TNM staging system was the only independent prognostic factor. In conclusion, although the IE of hK5/6/10/11 is down-regulated in RCC, tumors of high grade and/or advanced stage express one or more of the above kallikreins in a higher percentage. It seems that hK6 may significantly contribute to information in predicting poor disease outcome for RCC.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Below full citations are set out for the references referred to in the specification.

TABLE 1

| | Kallikrein | |
|---|---|---|
| Kallikrein | Nucleic | |
| Poly- | Acid | |
| peptide | Designation | GenBank Accession No. |
| Kallikrein | KLK5 | AAF03101, AF135028 (mRNA join |
| 5 | | 2101 . . . 2131, 2210 . . . 2293, |
| | | 4762 . . . 5023, 5763 . . . 6019, |
| | | 6105 . . . 6238, 11092 . . . 11570) |
| | | (CDS join 2221 . . . 2293, |
| | | 4762 . . . 5023, 5763 . . . 6019, |
| | | 6105 . . . 6238, 11092 . . . 11247), |
| | | AF168768 (CDS 170 . . . 1051) |
| Kallikrein | KLK6 | AFB66483, AF013988 (CDS 174 . . . 881), |
| 6 | | AF149289 (CDS 3567 . . . 3606, |
| | | 4346 . . . 4502, 8211 . . . 8369, |
| | | 9791 . . . 9927, 11805 . . . 11954) |
| | | (mRNA join 2001 . . . 2185, |
| | | 3084 . . . 3135, 3559 . . . 3606, |
| | | 4346 . . . 4502, 8122 . . . 8369, |
| | | 9791 . . . 9927, 11805 . . . 11957), |
| | | HSU62801 (CDS 246 . . . 980) |
| Kallikrein | KLK10 | AAC14266, AF055481 (CDS 614 . . . 701, |
| 10 | | 2455 . . . 2635, 3589 . . . 3863, |
| | | 4195 . . . 4328, 4793 . . . 4945) |
| | | (mRNA join 48 . . . 120, 605 . . . 701, |
| | | 2455 . . . 2635, 3589 . . . 3863, |
| | | 4195 . . . 4328, 4793 . . . 5474), |
| | | NM_002776 (CDS 220 . . . 1050) |
| Kallikrein | KLK11 | BAA33404, AAD47815, AB012917 |
| 11 | | (CDS 26 . . . 874), AF164623 |
| | | (CDS 4224 . . . 4263, 5061 . . . 5217, |
| | | 5545 . . . 5810, 6627 . . . 6727 . . . 6763, |
| | | 7158 . . . 7307((mRNA join |
| | | 2312 . . . 2398, 4189 . . . 4263, |
| | | 5061 . . . 5217, 5545 . . . 5810, |
| | | 6627 . . . 6763, 7158 . . . 7310) |

1. A method for detecting kallikrein polypeptides or polynucleotides encoding kallikrein polypeptides that are associated with renal cell carcinoma in a patient comprising: (a) taking a sample from a patient; (b) detecting or identifying in the sample one or more kallikrein polypeptides or polynucleotides encoding the kallikrein polypeptides wherein the kallikrein polypeptides comprise or are selected from the group consisting of kallikrein 5, kallikrein 6, kallikrein 10, and kallikrein 11; and (c) comparing the detected amount with an amount detected for a standard.

2. A method for detecting kallikrein polypeptides associated with renal cell carcinoma in a patient comprising: (a) obtaining a sample from a patient; (b) detecting in the sample one or more kallikrein polypeptides, wherein the kallikrein polypeptides comprise or are selected from the group consisting of kallikrein 5, kallikrein 6, kallikrein 10, and kallikrein 11; and (c) comparing the detected amounts with amounts detected for a standard.

3. A method for diagnosing and monitoring renal cell carcinoma in a subject comprising detecting in a sample from the subject one or more kallikrein polypeptides, wherein the kallikrein polypeptides comprise or are selected from the group consisting of kallikrein 5, kallikrein 6, kallikrein 10, and kallikrein.

4. A method as claimed in claim 1 wherein the kallikrein polypeptides are detected using antibodies that bind to the kallikrein polypeptides or parts thereof.

5. A method of detecting renal cell carcinoma in a patient, the method comprising comparing: (a) levels of kallikrein polypeptides in a sample from the patient, wherein the kallikrein polypeptides comprise or are selected from the group consisting of kallikrein 5, kallikrein 6, kallikrein 10, and kallikrein 11; and (b) normal levels of expression of the kallikrein polypeptides in a control sample, wherein a significant difference in levels of kallikrein polypeptides, relative to the corresponding normal levels, is indicative of renal cell carcinoma.

6. A method of claim 1 where the levels of kallikrein polypeptides are higher than normal levels and are indicative of advance disease and/or poor prognosis.

7. A method for monitoring the progression of renal cell carcinoma in a patient, the method comprising: (a) detecting in a sample from the patient at a first time point, kallikrein polypeptides, wherein the kallikrein polypeptides comprise or are selected from the group consisting of kallikrein 5, kallikrein 6, kallikrein 10, and kallikrein 11; (b) repeating step (a) at a subsequent point in time; and (c) comparing levels detected in steps (a) and (b), and thereby monitoring the progression of renal cell carcinoma.

8. A method for determining in a patient whether renal cell carcinoma has metastasized or is likely to metastasize in the future, the method comprising comparing (a) levels of kallikrein polypeptides, in a patient sample, wherein the kallikrein polypeptides comprise or are selected from the group consisting of kallikrein 5, kallikrein 6, kallikrein 10, and kallikrein 11; and (b) normal levels or non-metastatic levels of the kallikrein polypeptides, in a control sample wherein a significant difference between the levels of expression in the patient sample and the normal levels or non-metastatic levels is an indication that the renal cell carcinoma has metastasized.

9. A method for assessing the aggressiveness or indolence of renal cell carcinoma comprising comparing: (a) levels of expression of kallikrein polypeptides, in a patient sample, wherein the kallikrein polypeptides comprise or are selected from the group consisting of kallikrein 5, kallikrein 6, kallikrein 10, and kallikrein 11; and (b) normal levels of expression of the kallikrein polypeptides, in a control sample, wherein a significant difference between the levels in the patient sample and normal levels is an indication that the cancer is aggressive or indolent.

10. A method for diagnosing and monitoring renal cell carcinoma in a sample from a subject comprising isolating polynucleotides from the sample, and detecting in the sample polynucleotides encoding kallikrein polypeptides, wherein the kallikrein polypeptides comprise or are selected from the group consisting of kallikrein 5, kallikrein 6, kallikrein 10, and kallikrein.

11. A method as claimed in claim 10 wherein significant differences in the levels of the polynucleotides in the sample compared to a control is indicative of disease, disease stage, stage, and/or prognosis.

12. A method for determining the presence or absence of renal cell carcinoma in a subject comprising: (a) contacting a sample obtained from the subject with oligonucleotides that hybridize to polynucleotides encoding kallikrein polypeptides, wherein the kallikrein polypeptides comprise or are selected from the group consisting of kallikrein 5, kallikrein 6, kallikrein 10, and kallikrein 11; and (b) detecting in the sample levels of nucleic acids that hybridize to the polynucleotides encoding kallikrein polypeptides relative to a predetermined cut-off value, and therefrom determining the presence or absence of renal cell carcinoma in the subject.

13. A method as claimed in claim 12, wherein the nucleic acids are mRNA and the levels of nucleic acids are detected by polymerase chain reaction.

14. A method as claimed in claim 12, wherein the nucleic acids are mRNA and the amounts of mRNA are detected using a hybridization technique, employing oligonucleotide probes that hybridize to kallikrein polypeptides.

15. A method for assessing the potential efficacy of a test agent for inhibiting renal cell carcinoma in a patient, the method comprising comparing: (a) levels of kallikrein polypeptides, and/or polynucleotides encoding same in a first sample obtained from a patient and exposed to the test agent, wherein the kallikrein polypeptides comprise or are selected from the group consisting of kallikrein 5, kallikrein 6, kallikrein 10, and kallikrein 11, and (b) levels of the kallikrein polypeptides and/or polynucleotides encoding same in a second sample obtained from the patient, wherein the sample is not exposed to the test agent, wherein a significant difference in the levels of expression of the kallikrein polypeptides and/or polynucleotides encoding same in the first sample, relative to the second sample, is an indication that the test agent is potentially efficacious for inhibiting renal cell carcinoma in the patient.

16. A method of claim 15 wherein the first and second samples are portions of a single sample obtained from the patient.

17. A method of claim 15 wherein the first and second samples are portions of pooled samples obtained from the patient.

18. A method of assessing the efficacy of a therapy for inhibiting renal cell carcinoma in a patient, the method comprising comparing: (a) levels of kallikrein polypeptides and/or polynucleotides encoding same in a first sample obtained from the patient, wherein the kallikrein polypeptides comprise or are selected from the group consisting of kallikrein 5, kallikrein 6, kallikrein 10, and kallikrein 11, and (b) levels of the kallikrein polypeptides and/or polynucleotides encoding same in a second sample obtained from the patient following therapy, wherein a significant difference in the levels of expression of the kallikrein polypeptides and/or polynucleotides in the second sample, relative to the first sample, is an indication that the therapy is efficacious for inhibiting renal cell carcinoma in the patient.

19. A method of selecting an agent for inhibiting renal cell carcinoma in a patient the method comprising (a) obtaining a sample comprising cancer cells from the patient; (b) separately exposing aliquots of the sample in the presence of a plurality of test agents; (c) comparing levels of kallikrein polypeptides and/or polynucleotides encoding same in each of the aliquots, wherein the kallikrein polypeptides comprise or are selected from the group consisting of kallikrein 5, kallikrein 6, kallikrein 10, and kallikrein 11; and (d) selecting one of the test agents which alters the levels of kallikrein polypeptides and/or polynucleotides encoding same in the aliquot containing that test agent, relative to other test agents.

20. A method of inhibiting renal cell carcinoma in a patient, the method comprising (a) obtaining a sample comprising cancer cells from the patient; (b) separately maintaining aliquots of the sample in the presence of a plurality of test agents; (c) comparing levels of kallikrein polypeptides and/or polynucleotides encoding same in each of the aliquots, wherein the kallikrein polypeptides comprise or are selected from the group consisting of kallikrein 5, kallikrein 6, kallikrein 10, and kallikrein 11; and (d) administering to the patient at least one of the test agents which alters the levels of kallikrein polypeptides and/or polynucleotides encoding same in the aliquot containing that test agent, relative to other test agents.

21. A method of assessing the renal cell carcinoma cell carcinogenic potential of a test compound, the method comprising: (a) maintaining separate aliquots of renal cell carcinoma cells in the presence and absence of the test compound; and (b) comparing expression of kallikrein polypeptides and/or polynucleotides encoding same, in each of the aliquots, wherein the kallikrein polypeptides comprise or are selected from the group consisting of kallikrein 5, kallikrein 6, kallikrein 10, and kallikrein 11, and wherein a significant difference in levels of kallikrein polypeptides and/or polynucleotides encoding same in the aliquot maintained in the presence of the test compound, relative to the aliquot maintained in the absence of the test compound, is an indication that the test compound possesses renal cell carcinoma cell carcinogenic potential.

22. An in vivo method for imaging renal cell carcinoma comprising: (a) injecting a patient with agents that bind to kallikrein polypeptides, the agents carrying labels for imaging the renal cell carcinoma, and wherein the kallikrein polypeptides comprise or are selected from the group consisting of kallikrein 5, kallikrein 6, kallikrein 10, and kallikrein 11; (b) allowing the agents to incubate in vivo and bind to the kallikrein polypeptides; and (c) detecting the presence of the labels localized to the renal cell carcinoma.

23. A method of inhibiting renal cell carcinoma in a patient at risk for developing renal cell carcinoma, the method comprising inhibiting expression of genes encoding kallikrein polypeptides, wherein the kallikrein polypeptides comprise or are selected from the group consisting of kallikrein 5, kallikrein 6, kallikrein 10 and kallikrein 11.

24. A method of claim 1 comprising at least two of the kallikrein polypeptides.

25. A method of claim 1 comprising at least three of the kallikrein polypeptides.

26. A method of claim 22 where the kallikrein polypeptides are kallikrein 6 and kallikrein 11.

27. A method of claim 1 wherein the patient sample comprises serum obtained from the patient.

28. A kit for carrying out a method as claimed in claim 1.

29. A kit for assessing whether a patient is afflicted with renal cell carcinoma, the kit comprising reagents that specifically bind with kallikrein polypeptides, wherein the kallilcrein polypeptides comprise or are selected from the group consisting of kallikrein 5, kallikrein 6, kallikrein 10, and kallikrein 11.

30. A kit for assessing the suitability of each of a plurality of agents for inhibiting renal cell carcinoma in a patient, the kit comprising: (a) the plurality of agents; and (b) reagents for detecting kallikrein polypeptides, wherein the kallikrein polypeptides comprise or are selected from the group consisting of kallikrein 5, kallikrein 6, kallikrein 10, and kallikrein 11.

31. A kit as claimed in claim 28 wherein the reagents are antibodies that specifically bind with protein or protein fragments corresponding to kallikrein polypeptides.
